(19) United States
(12) Patent Application Publication
(10) Pub. No.: US 2015/0284674 A1
(43) Pub. Date: Oct. 8, 2015

(54) CAMPYLOBACTER BACTERIA AND DETECTION

(71) Applicant: THE SECRETARY OF STATE FOR ENVIRONMENT, FOOD & RURAL AFFAIRS, Surrey (GB)

(72) Inventors: Timothy Crawshaw, Surrey (GB); Jeremy Chanter, Surrey (GB); Adrian Whatmore, Surrey (GB); Mark Koylass, Surrey (GB); Ana Vidal, Surrey (GB); Richard Irvine, Surrey (GB)

(21) Appl. No.: 14/417,718

(22) PCT Filed: Aug. 2, 2013
(86) PCT No.: PCT/GB2013/052076

§ 371 Date: Jan. 27, 2015

(30) Foreign Application Priority Data
Aug. 2, 2012 (GB) 1213767.5

(51) Int. Cl. C12N 1/20; C07K 16/12; C07K 14/205

(52) U.S. Cl. CPC C12N 1/20; C07K 14/205; C07K 16/121

(57) ABSTRACT

There is provided a polynucleotide comprising a sequence at least 98.5% identical to SEQ ID NO: 1 and cells comprising this sequence, for example ECACC Deposit Reference 12022102. The polynucleotides and cells are useful in the detection of spotty liver syndrome and spotty liver disease.

### FIELD OF THE INVENTION

A novel bacterium is disclosed, isolated from samples obtained from chickens having spotty liver disease. A novel polynucleotide sequence is also disclosed, as well as antibodies and methods useful in the detection of the bacterium and of spotty liver disease.

### BACKGROUND

The terms spotty liver syndrome and spotty liver disease (SLS/SLD) have been used to describe a condition of domestic fowl that causes acute mortality with characteristic gross and microscopic pathology but of unknown aetiology. SLS/SLD cases have been reported in England, Scotland and Australia. The pathology and epidemiology are similar to the condition avian vibrionic hepatitis (AVH) which was first described in the 1950s (Tudor (1954) J. Am. Vet. Med. Assoc. vol 125 p 219). It is possible that they are the same condition and recently the terms have been used synonymously (Jennings et al. (2011) Vet. Microbiol. vol. 149 p 193-199).

SLS/SLD predominantly affects free-range laying hens around peak lay, but has also been recorded in caged layers and chickens reared for meat. The characteristic findings are multifocal 1-2 mm grey-white lesions in the liver (Crawshaw & Young (2003), Veterinary Record vol. 153 p 664). Histopathology shows an acute multifocal hepatic necrosis. A predilection for outbreaks to occur in the warmer months of the year has been noted in England and Australia where SLS/SLD is also called “summer hepatitis”.

Reports of cases have sometimes described a mild enteritis coincident with the onset of flock mortality. These reports mention that intestinal or caecal worms have also been present in some affected birds and they may be a predisposing factor.

The condition that was later described as AVH was first described in North America in the 1950s (Tudor (1954) J. Am. Vet. Med. Assoc. vol 125 p 219). It was shown to be transmissible and a Vibrio-like organism was isolated and suspected to be the cause. Experimental work at the time reproduced disease in day-old chicks (Delaplane (1955) Southwestern Veterinarian vol. 8 p 356; Winterfield (1957) Vet. Med. vol. 52 p 273) and 3 week old poults, but the organism was never fully defined. Attempts made in 1985 to propagate the agent from material stored since 1960 were unsuccessful (Clark (1986) Proc. 35^{th} Western Poultry Diseases Conference, p 25-27).

Reports of AVH affecting poultry flocks in North America continued until the 1960s and then declined. There have also been reports from Europe including Germany, Italy and Estonia. More recently there have been reports of AVH from Egypt, Taiwan and the Republic of Ireland. However, as there is no definitive test available it is not clear whether all these reports represent the same condition.

Campylobacter jejuni has been suggested to be the cause of AVH and it has been more frequently isolated from typically lesioned broiler livers than normal livers (Boukraa et al. (1991) Avian Dis. Vol. 35 p 714-717; Muller et al. (2011) Res. Vet. Sci. vol. 91 p 48-52), but it has not been possible to reproduce the disease experimentally using poultry or human derived strains of Campylobacter jejuni (Jennings et al. (2011) Vet. Microbiol. vol. 149 p 193-199). Helicobacter pullorum has also been isolated from cases of AVH (Stanley et al. (1994) Microbiology vol. 140 (Pt 2) p 3441-3449) though there is no evidence to suggest it is involved in the aetiology.

There remains a need to identify the causative organism for SLS/SLD and to develop assays to detect and diagnose this condition, as well as to develop a vaccine.

### SUMMARY OF THE INVENTION

The inventors have isolated and characterized a novel Campylobacter from five outbreaks of SLS/SLD that occurred in four free-range laying flocks in England. The novel Campylobacter was also confirmed as present in birds in an outbreak in a fifth flock.

According to a first aspect of the invention there is provided a polynucleotide comprising a sequence 1290 nucleotides in length which is at least about 98.5% identical to the sequence SEQ ID NO:1, or at least about 99.0%, 99.1%, 99.2%, 99.3%, 99.4%, 99.5%, 99.6%, 99.7%, 99.8% or at least about 99.9% identical. For example, the sequence may be SEQ ID NO:5, which differs from SEQ ID NO:1 in having nucleotide A at position 1141 (of SEQ ID NO:1), instead of nucleotide G. SEQ ID NO:5 is therefore more than 99.9% identical to SEQ ID NO:1.

The sequence may be isolated and, in an embodiment, may be a synthetic (i.e., laboratory-generated) or recombinant sequence such as a cDNA. SEQ ID NO:1 has been identified as the conserved 1290 bp fragment of the 16S rRNA gene sequence obtained from bacterial isolates cultured from the SLS/SLD events mentioned above. Phylogenetic analysis and comparison of sequences with equivalent regions of the type strains of all currently described Campylobacter species revealed that the isolates represent a hitherto unknown sublineage within the group. Pairwise sequence comparisons over the same 1290 bp sequence revealed similarities of up to 98.45% with type strains of some Campylobacter species, as outlined further below. Levels of similarity with other Campylobacter species were substantially lower.

The polynucleotide according to the invention is at least about 98.5% identical to a sequence consisting of SEQ ID NO:1, i.e., the sequence identity determination is to be made in comparison with the whole length of SEQ ID NO:1, not a smaller fragment thereof.

Percentage sequence identity may be determined using a global sequence alignment tool such as the Needleman-Wunsch Global Sequence Alignment Tool available from the National Center for Biotechnology Information (NCBI), Bethesda, Md., USA, for example via http://blast.ncbi.nlm.nih.gov, using default parameter settings. The Needleman-Wunsch algorithm was published in J. Mol. Biol. (1970) vol. 48:443-53. As mentioned, when comparing the level of sequence identity to SEQ ID NO:1, this typically should be done relative to the whole length of SEQ ID NO:1, to avoid short regions of high identity overlap resulting in a high overall assessment of identity (i.e., a global alignment method is used). For example, a short polynucleotide fragment having, for example, fifteen nucleotides might have a 100% identical sequence to a fifteen nucleotide region within the whole of SEQ ID NO:1, but this does not provide a 100% sequence identity unless the fragment forms part of a longer sequence which also has identical nucleotides at the other positions equivalent to positions in SEQ ID NO:1.

Polynucleotide sequences which are the complementary sequence to SEQ ID NOs:1 or 5, or variants which are at least 98.5% identical to such a complementary sequence, are also contemplated and encompassed by the first aspect of the present invention. The RNA sequence encoded by SEQ ID NOs:1 or 5 (i.e., SEQ ID NOs:1 or 5 can be transcribed to the RNA sequence) and variants which are at least 98.5% identical thereto are also contemplated and encompassed by the first aspect of the invention.

In an embodiment, the polynucleotide comprises the sequence SEQ ID NO:2, or a complementary sequence thereof. SEQ ID NO:2 is the 1436 bp 16S rRNA sequence discussed further below, which comprises SEQ ID NO:1 (SEQ ID NO:1 corresponds to nucleotides 53-1342, inclusive, of SEQ ID NO:2). The sequence may be isolated and, in an embodiment, may be a laboratory-generated sequence such as a cDNA.

Any of the cDNA or RNA sequences mentioned herein may comprise alterations to SEQ ID NO:1 or 2 or 5 in order to codon-optimise the sequence for expression in a host cell which is not a bacterium.

The polynucleotide may be an expression vector comprising SEQ ID NO:1 or 2 or 5 and expression control elements, such as promoter sequences and selection cassettes (such as antibiotic or antifungal sequences). The construction of such an expression vector is within the routine ability of the skilled person (see, for example, Green & Sambrook; Molecular Cloning, a laboratory manual [fourth edition] Green & Sambrook, Cold Spring Harbor Laboratory, 2012).

In a second aspect of the invention, there is provided a polynucleotide at least 6 nucleotides in length, capable of hybridising to a novel portion or region of a polynucleotide according to the first aspect of the invention. That is, these polynucleotides (called “probe polynucleotides” herein, but intended to encompass amplification primers as well as hybridisation probes) are capable of hybridising to SEQ ID NO:1 or 5 but not to a sequence which is not SEQ ID NO:1 or 5. Such a probe polynucleotide may, therefore, hybridise to a region of SEQ ID NO:1 or 5 comprising a nucleotide which differs to the nucleotide in the equivalent position in a 16S sequence which is not SEQ ID NO:1 or 5.

The nucleotides at the following positions of SEQ ID NO:1 differ compared to the nucleotides at the equivalent positions of the 16S gene sequence in other Campylobacter, i.e., the nucleotides in these positions are unique in SEQ ID NOs:1 and 5. They are conserved in SEQ ID NOs:1 and 5, but differ in Campylobacter 16S gene sequences which are not one of these sequences.

| SEQ ID NO: 1 | Nucleotide at | Nucleotide in other |
|---|---|---|
| nucleotide | that position in | Campylobacter 16S |
| position | SEQ ID NOs: 1 & 5 | sequences |
| 168 | T | C |
| 196 | A | G |
| 375 | T | C |
| 554 | A | G |
| 558 | T | G |
| 637 | A | G |
| 1152 | A | G |
| 1195 | T | C |
| 1196 | T | C |

For example, a sequence might differ from SEQ ID NO:1 in that the 168^{th} position nucleotide is “T” instead of “C”; in this case, the probe polynucleotide of the invention would hybridise to SEQ ID NO:1 in a region comprising this position, but would not hybridise to the sequence comprising “C” instead of “T” at the 168^{th} position.

Therefore, the probe polynucleotide may specifically hybridise to a portion of SEQ ID NO:1 which comprises one or more of the nucleotides unique to SEQ ID NOs:1 and 5 at positions 168, 196, 375, 554, 558, 637, 1152, 1195 or 1196 (as outlined above), with nucleotide position numbering in accordance with the numbering of SEQ ID NO:1. That is, the probe polynucleotide specifically hybridises to a portion of SEQ ID NO:1 which comprises one or more of 168T, 196A, 375T, 554A, 558T, 637A, 1152A, 1195T or 1196T. By way of non-limiting example, the probe may hybridise to a sequence comprising one or more of the following sequences:

| | Encompassing position no. | SEQ ID |
|---|---|---|
| Sequence | (shown as bold in sequence) | NO |
| TCAGTTAGTT | 168 | 6 |
| CAAGACTATG | 196 | 7 |
| AATTTTGACG | 375 | 8 |
| GAGTAAGGTA | 554 & 558 | 9 |
| AAGGTAGAGG | 554 & 558 | 10 |
| AGTAAGGTAG | 554 & 558 | 11 |
| TGCTAGAACT | 637 | 12 |
| CAATAAGACG | 1152 | 13 |
| GCATATACAATA | 1152 | 14 |
| ACATATACAATA | 1152 | 15 |
| TGTCTTAGTT | 1195 & 1196 | 16 |

The region of probe hybridisation must overlap with one or more of the variant positions mentioned above which is unique in SEQ ID NOs:1 and 5 compared to other Campylobacter 16S gene sequences. That is, a probe which will hybridise to one of SEQ ID NOs:6-16 without overlapping with the nucleotide highlighted in bold is not encompassed by the present invention.

The first nucleotide in each of SEQ ID NOs:14 and 15 (G and A, respectively) represents the alternatives in SEQ ID NOs:1 and 5 at position 1141. Therefore, a probe polynucleotide according to the invention may hybridise to one, or the other, or both sequences comprising either G or A at position 1141, provided the region of hybridisation also comprises one of the variant positions mentioned above.

The hybridisation is specific, in that it occurs with greater affinity than hybridisation of a probe polynucleotide according to the second aspect of the invention to a polynucleotide which is not according to the first aspect of the invention, as outlined above. For example, the Tₘ of a hybrid between polynucleotides according to the second and first aspects of the invention may be greater than the Tₘ of a hybrid between a probe polynucleotide according to the second aspect of the invention and a polynucleotide not of this invention. The probe polynucleotide according to the second aspect of the invention may comprise or consist of, for example, at least about 7, 8, 9, 10, 11, 12, 13, 14, 15, 16, 17, 18, 19, 20, 21, 22, 23, 24, 25, 26, 27, 28, 29, 30, 35, 40, 45 or at least about 50 nucleotides. For example, the probe polynucleotide may have up to about 100, 200, 300, 400, 500, 600, 700, 800, 900 or about 1000 nucleotides. Such a probe polynucleotide may, for example, be useful as an amplification primer or a hybridisation probe and, therefore, may be useful in detecting the presence in a sample of a polynucleotide according to the first aspect of the invention and, therefore, of a cell containing the polynucleotide.

The optimal length of such a primer or probe is readily determined by the skilled person, for example, using the software “Primer Express®” (Applied Biosystems). Various such software packages exist and are in regular use by the skilled person. A method of preparing or designing a probe polynucleotide which will specifically hybridise to SEQ ID NO:1 and/or 5, as described above, is encompassed by the present invention. The primer or probe may be labelled with a detectable label, for example a label which forms part of a visible signalling system which may, for example, utilise agents in particular dyes, that intercalate or otherwise interact with nucleic acids and generate a differential signal such as a fluorescent signal, depending upon whether or not they are interacting with a nucleic acid. Examples of such dyes include SYBR Green™ and SYTO dyes, by way of non-limiting example.

Certain visible signalling systems utilise oligonucleotide probes or primers that bind specific target nucleic acids and are labelled with a visible label such as a fluorescent label. Examples of suitable fluorescent labels are well understood by the skilled person and include, by way of non-limiting example, fluorescein or fluorescein derivatives such as FAM (5 or 6-carboxyfluorescein).

Signalling systems of this type may also involve the use of so called “quencher” moieties. These interact with fluorophores and absorb fluorescent energy from them, so they act as acceptors of fluorescent energy. If the system is arranged so that the physical separation of the fluorophore and the quencher differs depending upon the presence in a sample of a nucleic acid amplification product, the change in fluorescence can be used to characterise the amplification. Systems of this kind and their many variations are also very well known to and understood by the skilled person. Where a signalling system relies on fluorescence energy transfer between donor and acceptor or quencher moieties, these are often referred to as FET or FRET systems.

More than one probe may be utilised in such a signalling system, the first of which must be a probe polynucleotide according to the second aspect of the invention. The second probe may hybridise to a region near the hybridisation region of the first probe, to facilitate signalling interactions between labels on each probe. This second probe may also be according to the second aspect of the invention, or may be one which can hybridise to sequences other than SEQ ID NO:1 and/or 5, in addition to being capable of hybridising to SEQ ID NO:1 and/or 5.

The term “capable of hybridising to a polynucleotide according to the first aspect of the invention” indicates that the polynucleotide according to the second aspect of the invention (which may also be referred to as a “probe sequence”) substantially hybridises to the polynucleotide sequence of the present invention. Such hybridisation may occur at or between low and high stringency conditions. In general terms, low stringency conditions can be defined as hybridisation in which the washing step takes place in a 0.330-0.825M NaCl buffer solution at a temperature of about 40-48° C. below the calculated or actual melting temperature (Tₘ) of the probe sequence (for example, about ambient laboratory temperature to about 55° C.), while high stringency conditions involve a wash in a 0.0165-0.0330M NaCl buffer solution at a temperature of about 5-10° C. below the calculated or actual Tₘ of the probe sequence (for example, about 65° C.). The buffer solution may, for example, be SSC buffer (0.15M NaCl and 0.015M tri-sodium citrate), with the low stringency wash taking place in 3×SSC buffer and the high stringency wash taking place in 0.1×SSC buffer. Steps involved in hybridisation of nucleic acid sequences have been described for example in Green & Sambrook (2012).

A third aspect of the invention provides an amplification system comprising at least one amplification primer (for example, SEQ ID NO:3 or 4) and at least one probe polynucleotide according to the second aspect of the invention, as described above. Such an amplification system may be suitable for use with, for example, polymerase chain reaction (PCR). The at least one amplification primer may also be one capable of hybridising to SEQ ID NO:17 or 18, which are the end regions of SEQ ID NO:2 flanking SEQ ID NO:1 (see FIG. 2). This enables amplification and detection of the 1290 bp region or the 16S gene which is conserved in the novel Campylobacter according to the invention.

According to a fourth aspect of the invention, there is provided a bacterial cell comprising a polynucleotide sequence according to the first aspect of the invention. Such a cell may be a member of the new Campylobacter sublineage disclosed herein. In an alternative embodiment, the cell may be a non-Campylobacter species comprising a polynucleotide sequence according to the first aspect of the invention; for example, the cell may be an Escherichia coli cell or a yeast cell such as a Saccharomyces cerevisiae cell. A fifth aspect of the present invention provides a bacterial cell deposited as ECACC Deposit Reference 12022102. Such a cell may comprise a polynucleotide sequence according to the first aspect of the invention.

The cell according to the fourth and fifth aspects of the invention may exhibit one or more of the following characteristics:
a) oxidase activity;
b) catalase activity;
c) microaerobic growth at about 37.0° C.±1° C. or 41.5° C.±1° C.;
d) susceptibility to nalidixic acid;
e) γ-glutamyltranspeptidase activity;
f) alkaline phosphatase activity;
g) very weak or no ability to hydrolyse hippurate;
h) very weak or no ability to hydrolyse indoxyl acetate;
i) motile curved bacteria under microscopic examination; and/or
j) unable to grow in aerobic conditions.

These properties may be determined, for example, using the methods described in the Examples section below. These properties may particularly be present when the cell is a Campylobacter cell and may not be present when the cell is a non-Campylobacter cell such as an Escherichia coli cell, or a yeast cell such as a Saccharomyces cerevisiae cell.

The cell according to the fourth or fifth aspects of the invention may be modified from a naturally occurring cell by being attenuated (to a live but less virulent cell, i.e., a cell less capable or not capable of causing disease) or by being inactivated (killed). In such a case, the cell may be a Campylobacter cell. Such a cell may be useful to vaccinate an animal, such as a bird or a mammal, against developing SLS/SLD and/or against infection by a cell according to the fourth or fifth aspects of the invention.

The cell may be attenuated or inactivated by exposure to an inactivation agent or chemical. Vaccines derived from inactivated strains of bacteria are well known in the art. The pathogenic microorganism is treated with a chemical, which damages the organism to such an extent that it cannot cause infective disease in an animal to which it is administered. For example, the ability of the organism to replicate may be irreparably damaged. However, key antigenic regions of the organisms are retained, which stimulate an immune response in the animal to which they are administered. This response may provide a protective effect if the animal is later challenged with a potent (i.e., more virulent) strain of the organism.

Different inactivating agents have different modes of action in order to achieve the desired effect. Some agents bind DNA, others destroy or inactivate essential proteins and a further class of inactivating agents may disrupt the cell membrane. The efficacy of the agents in any particular case will depend upon the nature of the microorganism. Thus, the results achieved will be variable, as a result of the inherent variability of the microorganisms.

Examples of typical inactivating agents include formaldehyde, which is usually used in the form of a solution known as ‘formalin’, azide, detergents (especially non-ionic detergent), lysozyme, phenol, proteolytic enzymes, propiolactone such as beta-propiolactone (BPL), sodium ethyl mercury thiosalicylate (Thimerosal) and binary ethyleneimine.

In another embodiment, inactivation may be effected by an agent that disrupts cell membranes and, in particular, only partially disrupts cell membranes. A particular example of such an inactivation agent is saponin. By partially disrupting cell membranes with saponin, the cells are prevented from growing, yet may retain the key antigenic regions of the organism, which stimulate an immune response in the animal.

As used herein, the term “saponin” refers to sapogenin glycosides, which may be derived from plants such as Quillaga bark. These compounds are known to have haemolytic activity and may be used as adjuvants for vaccines. When vaccines are produced by inactivation using saponin, there is no need to subsequently separate the strain from any residual saponin, since this may act as an adjuvant. If desired, further saponin may be added to any vaccine composition obtained in this way, in order to enhance the adjuvant effect.

Attenuation of a live cell may be achieved by, for example, serial passage of the cells through culture media. Genetic modification may also be utilised to achieve a cell which is alive but with reduced virulence (i.e., ability to cause disease) compared to the non-attenuated cell.

A sixth aspect of the invention provides the genome sequence of the cell according to the fifth aspect of the invention (ECACC Deposit 12022102) and novel portions thereof, as well as cDNA obtained using the genome sequence and novel portions thereof. The genome comprises SEQ ID NOs:1 and 2, or 5. At least one other gene forming a portion of the genome differs from the closest known gene sequence by at least about 10%, i.e., the closest related prior art gene sequence identified using sequence comparison software (such as the nucleotide BLAST software available via http://blast.ncbi.nlm.nih.gov) is no more than about 90% identical to the sequence from the present bacterium. Alternatively or additionally, a comparison at whole genome level may result in sequence identity (for the whole genome) of no more than about 90%. Polynucleotides at least 6 nucleotides in length (or longer, as set out for the second aspect of the invention), capable of hybridizing to novel portions of the genome sequence or portion thereof of the cell of the invention are also contemplated. Such a polynucleotide may be useful as an amplification primer or a hybridisation probe and, therefore, may be useful in detecting the presence in a sample of a cell according to the invention. The primers and probes may be utilised with amplification and signalling systems as described elsewhere herein.

According to a seventh aspect of the invention, there is provided an antibody or antibody mimetic capable of binding to a polynucleotide according to the first aspect of the invention and/or to a cell according to the fourth and/or fifth aspects of the invention, as well as a functional fragment of such an antibody or antibody mimetic. The binding may be specific, which indicates that the antibody or antibody mimetic recognises and binds to the polynucleotide and/or cell according to the invention in preference to other polynucleotides and/or cells not according to the invention.

The term “antibody mimetic” as used throughout this specification indicates a component of a molecular recognition system capable of the specific binding mentioned in the previous paragraph. The term includes, by way of non-limiting example, synthetic antibodies (“Synbodies”), oligonucleic acid aptamers, peptide aptamers and Affibody® molecules.

An antibody or antibody mimetic capable of binding to a polynucleotide according to the first aspect of the invention may have been raised against such a polynucleotide. Likewise, an antibody or antibody mimetic capable of binding to a cell according to the fourth and/or fifth aspects of the invention may have been raised against such a cell or an immunogenic portion thereof. The antibody may be produced by hybridoma cell lines or may also be produced by recombinant genetic methods well known to a person skilled in the art. The antibody may be animal derived, typically a mouse antibody as well as a human antibody, a chimeric antibody, a “humanized antibody”, a primatized antibody, etc.

Fragments of the antibodies or antibody mimetics which fall within the scope of the present invention are any which substantially retain the binding characteristics of the whole antibodies or antibody mimetics from which they are derived and thus specifically bind to the polynucleotide and/or the cell of the invention.

The term “substantially retain the binding characteristics” should be understood to mean that the antibody fragment or antibody mimetic fragment has a binding affinity for the polynucleotide and/or the cell of the invention of at least about 50% of the binding affinity of the whole antibody or antibody mimetic for the same polynucleotide and/or cell.

Hybridoma cell lines which produce the mAb of the invention are also within the scope of the invention. Such hybridomas may be prepared by any of the methods known in the art such as described in, e.g., Kohler and Milstein, Nature, 256: 495-497, (1975). The present invention further provides mouse hybridoma cell lines which produce any of the monoclonal antibodies of the invention. The supernatants of the hybridoma cell lines are typically screened for antibody binding activity by any one of the methods known in the art such as by enzyme linked immunosorbent assay (ELISA) or radio immunoassay (RIA). The supernatants are screened for production of mAbs capable of binding to a polynucleotide and/or a cell according to the invention.

The present invention further provides recombinant cell lines or transgenic animals expressing antibodies and/or antibody mimetics of the invention.

Various hosts can be used for production of antibodies by the hybridoma technique including rats, mice, etc. The animals may be immunized by injecting the polynucleotide and/or cell of the invention to the host. Various adjuvants may be used to increase the immunological response such as Freund's adjuvant, mineral gels, aluminium hydroxide, etc.

In addition to the hybridoma technique mentioned above, continuous cell lines which produce antibodies obtained by additional techniques may also be used such as, for example, the EBV-hybridoma technique (Cole et al. (1984) Mol. Cell Biol., 62: 109).

Antibodies may also be produced by inducing in vivo production in the appropriate lymphocyte population or by screening recombinant immunoglobulin libraries in accordance with known methods which are described, for example, in Orlandi et al. (1989) Proc. Natl. Acad. Sci. USA vol. 86 p. 3833-3837.

Antibodies may be monoclonal or polyclonal, and are preferably monoclonal, but the term “antibody” also encompasses binding fragments of antibodies such as Fab, F(ab′)₂ fragments or single chain antibody fragments.

According to an eighth aspect of the invention, there is provided a kit comprising a polynucleotide according to the first aspect of the invention and/or a polynucleotide according to the second aspect of the invention and/or an amplification system according to the third aspect of the invention and/or a polynucleotide according to the sixth aspect of the invention and/or an antibody or antibody mimetic according to the seventh aspect of the invention. Such a kit may comprise instructions for use, for example, to enable detection of a polynucleotide according to a first aspect of the invention and/or a cell according to a fourth or fifth aspects of the invention in a sample.

The kit may also comprise reagents appropriate to its intended use. For example, where the kit comprises a probe polynucleotide according to the second aspect or an amplification system according to the third aspect of the invention, the kit may also comprise PCR assay reagents selected from, for example, amplification primers, a PCR buffer solution, a MgCl₂ solution, a polymerase enzyme and one or more dNTPs. Other possible kit components, for example reagents for other non-PCR assay systems, may be envisaged by the skilled person.

A ninth aspect of the invention provides a method of detecting the presence of a cell according to the fourth or fifth aspects of the invention in a sample, comprising:
a) detecting the presence in the sample of a polynucleotide according to the first or sixth aspects of the invention in the sample; and/or
b) detecting the presence in the sample of an antibody according to the seventh aspect of the invention; and/or
c) contacting the sample with an antibody or antibody mimetic according to the seventh aspect of the invention, the antibody or antibody mimetic being capable of binding to a cell according to the fourth or fifth aspects of the invention, and detecting the antibody or antibody mimetic binding to such a cell, wherein detection of binding is indicative of the presence of the cell in the sample.

Where the method involves detecting the presence in the sample of a polynucleotide according to the first or sixth aspects of the invention in the sample, the detection may be a nucleic acid amplification method such as polymerase chain reaction, or an isothermal amplification method, such as nucleic acid sequence based amplification (NASBA), strand displacement amplification (SDA), transcription mediated amplification (TMA), Loop-Mediated Isothermal Amplification (LAMP), Q-beta replicase, Rolling circle amplification, 3SR, ramification amplification (as described by Zhang et al., Molecular Diagnosis (2001) vol. 6, p 141-150) and others. The method may comprise use of an amplification system according to the third aspect of the invention and/or a kit according to the eighth aspect.

Alternatively or additionally, the method may comprise a nucleic acid hybridisation method, where hybridisation of a labelled probe polynucleotide to the target nucleic acid sequence is indicative of the presence of the target sequence in the sample. A further alternative in this method may comprise an immunoassay method, comprising binding of an antibody or antibody mimetic to a polynucleotide according to a first aspect of the invention and detection of such binding by means of, for example, an ELISA, or a FRET method as described in WO2009/118570 and WO2011/030168. The detection of a polynucleotide may also be by 16S rRNA sequencing, as disclosed herein.

These embodiments of the invention may comprise, as a preliminary step, a step of causing lysis of a cell according to the fourth aspect of the invention present in the sample, so as to release the polynucleotide to be detected. Alternatively or additionally, the method may comprise a preliminary process of extraction of nucleic acids contained within the cells in the sample, with the detection step (a) above being conducted on the sample resulting from the nucleic acid extraction process. Such methods are well known to the skilled person and may be as described, for example, in Green & Sambrook. (2012, as above).

As outlined above, an alternative embodiment of the ninth aspect of the invention comprises detecting the presence of an antibody according to the seventh aspect of the invention in the sample, or contacting the sample with an antibody or antibody mimetic according to the seventh aspect of the invention, the antibody or antibody mimetic being capable of binding to a cell according the fourth and/or fifth aspects of the invention, and detecting the antibody or antibody mimetic binding to such a cell, wherein detection of binding is indicative of the presence of the cell in the sample. Again, the detection of antibody in the sample, or of antibody or antibody mimetic binding may typically be by means of an assay such as an ELISA, or a FRET method as described in WO2009/118570 and WO2011/030168. When detection is by contacting the sample with an antibody or antibody mimetic, the antibody or antibody mimetic is typically specific for the cell according to the fourth and/or fifth aspects of the invention, in that it will bind to the cell according to the invention, but not to other cells or components of the sample.

The sample may be a liquid sample such as a water sample, for example a sample of waste or ground run-off water from a farm which houses animals which may be affected by SLS/SLD. Alternatively, the sample may be a biological sample, such as a tissue sample (for example, a liver sample), a blood sample, a saliva sample, a faecal sample, a urine sample, a bile sample, or any other type of sample obtained directly from an animal which may be affected by SLS/SLD. Reference to “sample” throughout this specification may be reference to any one or more of any of these types of sample. A sample may be subjected to preparatory processes prior to being contacted with the antibody or antibody mimetic. For example, a tissue sample may be a formalin-fixed sample. Reference to “animal” throughout this specification will typically indicate an avian animal such as a chicken, but may also indicate a mammal such as a human or a non-human mammal.

The polynucleotide according to the first or second or sixth aspects of the invention and/or the amplification system according to the third aspect of the invention and/or an antibody or antibody mimetic according to the seventh aspect of the invention and/or a kit according to the eighth aspect of the invention may be for use in a method of diagnosing SLS/SLD in an animal and/or for use in a method of detecting a cell according to the fourth or fifth aspects in a sample obtained from an animal. The method may be according to the ninth aspect, as outlined above. Similarly, a tenth aspect of the invention provides a method of diagnosing SLS/SLD in an animal comprising obtaining a biological sample from the animal and detecting the presence of a polynucleotide according to the first or sixth aspects of the invention and/or detecting the presence of a cell according to the fourth aspect of the invention and/or detecting the presence of an antibody according to the seventh aspect of the invention in the sample. For example, this may be achieved by use of the method according to the ninth aspect of the invention, as described above.

According to an eleventh aspect of the invention, there is provided a vaccine composition comprising a cell according to the fourth or fifth aspects of the invention, or comprising an immunogenic portion of such a cell. An immunogenic portion may be a fragment of the cell itself, or may be a polypeptide or polynucleotide component of such a cell. For example, a suitable polypeptide may be a surface protein of the cell, such as an O-antigen; a suitable polynucleotide may be one which encodes such a surface protein. The polynucleotide may be a portion of the polynucleotide forming the sixth aspect of the invention.

In a particular aspect, the vaccine composition comprises an attenuated or inactivated (i.e., killed) cell according to the fourth or fifth aspects of the invention, or an immunogenic portion thereof. The cell may, for example, have been attenuated or inactivated using one of the agents mentioned above.

The vaccine composition may be for use in vaccinating an animal, such as a bird or a mammal, against developing SLS/SLD and/or against infection by a cell according to the fourth or fifth aspects of the invention. The bird may be any domesticated bird, for example a chicken. The mammal may be a human.

The composition may further comprise a pharmaceutically or veterinarily acceptable carrier, which may be solid or liquid, depending upon the intended mode of administration of the vaccine. The vaccine may be intended for parenteral administration. Liquid carriers, such as sterile aqueous or oily carriers for intravenous, subcutaneous, or intramuscular dosing may, therefore, be preferred.

Thus, the compositions of the present invention suitably may be in the form of a sterile injectable aqueous or oily suspension, which may be formulated according to known procedures using one or more of the appropriate dispersing or wetting agents and suspending agents. A sterile injectable preparation may also be a sterile injectable solution or suspension in a non-toxic parenterally-acceptable diluent, for example a suspension in 1,3-butanediol.

Examples of suspending agents which may be used in the formulation of the invention include sodium carboxymethylcellulose, methylcellulose, hydroxypropylmethyl cellulose, sodium alginate, polyvinyl-pyrrolidone, gum tragacanth and gum acacia. The aqueous suspensions may also contain one or more preservatives (such as ethyl or propyl p-hydroxybenzoate or anti-oxidants (such as ascorbic acid).

Oily suspensions may be formulated by suspending the active ingredient in a vegetable oil (such as arachis oil, olive oil, sesame oil or coconut oil) or in a mineral oil (such as liquid paraffin).

Dispersible powders and granules suitable for preparation of an aqueous suspension by the addition of water generally contain the active ingredient together with a dispersing or wetting agent, suspending agent and one or more preservatives.

The amount of active ingredient that is combined with one or more excipients to produce a single dosage form will necessarily vary depending upon the host treated and the particular route of administration. The size of the dose for vaccination purposes will naturally vary according to the nature, the age of the animal and the route of administration, according to well-known principles of medicine.

The composition of the invention may comprise further components, which would be known in the art. They may, for example include adjuvants such as aluminium compounds or Freund's incomplete adjuvant. Saponin, when present in the composition as a result of its use as an inactivating agent, will, however, also have an adjuvant effect and it is unlikely, therefore, that further adjuvants would be required.

In any embodiment, the vaccine composition may form a component of a multivalent vaccine composition.

A twelfth aspect of the invention provides a feedstock comprising a vaccine composition according to the eleventh aspect of the invention. For example, where the vaccine composition is to be provided to a chicken, the feedstock may comprise a grain-based feed.

A thirteenth aspect of the invention provides a method of vaccinating an animal against developing SLS/SLD and/or against infection by a cell according to the fourth or fifth aspects of the invention, the method comprising administering an effective amount of the vaccine composition according to the eleventh aspect and/or the feedstock according to the twelfth aspect of the invention. The term “effective amount” indicates that sufficient composition is administered that the probability of the animal developing SLS/SLD and/or developing infection by the cell is reduced or obviated.

A fourteenth aspect of the invention provides a method of preparing a vaccine composition according to the eleventh aspect of the invention, comprising exposing a cell according to the fourth or fifth aspects of the invention to an inactivating agent as described above and, optionally, mixing the resulting cell with a pharmaceutically or veterinarily acceptable carrier as described above.

A fifteenth aspect of the invention provides a method of isolating a Campylobacter strain from a test sample, comprising a step of spreading an amount of a mixed sample obtained from the test sample onto a solid medium comprising sheep's blood, wherein the sample has not previously been subjected to an earlier step of spreading onto a solid medium which comprises sheep's blood. A test sample may be one which is directly obtained from an animal (for example, it may be a liver biopsy or other sample such as a liquid bile sample from a bird suspected to be suffering from SLS/SLD) or it may be an environmental sample, such as a water or a soil sample.

The mixed sample may either be the test sample itself, especially when the test sample is obtained in liquid form, or it may be obtained by mixing the test sample with a liquid such as a bacterial growth medium as described herein. Where the test sample is a solid sample such as a liver biopsy, the mixed sample may be obtained by breaking the solid material up, for example by maceration, in a liquid such as a bacterial growth medium.

The method may be especially suitable for obtaining (i.e., isolating) a Campylobacter strain such as a cell according to the fourth or fifth aspects of the present invention.

The solid medium comprising sheep's blood may be, for example, an agar medium comprising 2-10% sheep's blood, for example about 5% sheep's blood. The agar medium may be contained within a conventional petri dish, as is well known in the art.

The method may comprise a step of incubating the solid medium at 35-42° C. for at least about 2 days after the mixed sample has been spread on the solid medium (for example, using a process as described in more detail below). The incubation may be, for example, at about 35° C., 36° C., 37° C., 38° C., 39° C., 40° C., 41° C. or about 42° C. Deviation from the intended temperature by up to about 1° C., 2° C., 3° C., 4° C. or up to about 5° C. may be acceptable. Independently, the incubation period may be selected from at least about 2, 3, 4, 5, 6, 7, 8, 9 or at least about 10 days (±12 hours), typically up to around 7 days (±12 hours).

In the method, the step of spreading an amount of the mixed sample onto the solid medium may be preceded by obtaining the test sample, forming the mixed sample by mixing the test sample with a liquid growth medium such as Preston broth, and incubating the mixed sample at 35-42° C. for at least about 2 days. Again, the incubation may be, for example, at about 35° C., 36° C., 37° C., 38° C., 39° C., 40° C., 41° C. or about 42° C. Again, deviation from the intended temperature by up to about 1° C., 2° C., 3° C., 4° C. or up to about 5° C. may be acceptable. Independently, the incubation period may again be selected from at least about 2, 3, 4, 5, 6, 7, 8, 9, or at least about 10 days (±12 hours), typically up to around 7 days (±12 hours). The test sample may be obtained under conditions designed to minimise the risk of contamination by other organisms, for example by use of sterile sampling apparatus.

Using the novel method according to this last aspect of the invention, the inventors isolated the novel Campylobacter strain described herein, for the first time. This important breakthrough provides new detection and vaccination tools to combat SLS/SLD.

Throughout the description and claims of this specification, the words “comprise” and “contain” and variations of the words, for example “comprising” and “comprises”, mean “including but not limited to” and do not exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Other features of the present invention will become apparent from the following examples. Generally speaking the invention extends to any novel one, or any novel combination, of the features disclosed in this specification (including the accompanying claims and drawing). Thus, features, integers, characteristics, compounds or chemical moieties described in conjunction with a particular aspect, embodiment or example of the invention are to be understood to be applicable to any other aspect, embodiment or example described herein, unless incompatible therewith.

Moreover, unless stated otherwise, any feature disclosed herein may be replaced by an alternative feature serving the same or a similar purpose.

### BRIEF DESCRIPTION OF THE FIGURES

The invention will now be described, by way of example only, with reference to FIGS. 1-9 in which:

FIG. 1 shows a phylogenetic analysis comparing the SLS/SLD isolates from farms 1-4 with type strains of all currently described Campylobacter species, inferred from 16S rRNA gene comparison. The tree was constructed in MEGA5 (http://www.megasoftware.net) using the neighbour-joining approach following CLUSTAL alignment of sequences trimmed to a 1192 bp consensus contig and applying the Jukes-Cantor substitution mode and the pairwise deletion option. Numbers at nodes correspond to proportions of 500 resamplings that support the topology shown with values >50% indicated. Bar=0.01 substitutions per nucleotide position. The sequence of Helicobacter pylori was used as an outgroup;

FIG. 2 shows SEQ ID NO:2, with SEQ ID NO:1 underlined;

FIG. 3 is a MALDI dendrogram showing phyloproteomic relationship of novel Campylobacter strains with other Campylobacter species and related members of the taxa;

FIG. 4 (A, B, C) shows electron micrographs of strains of novel Campylobacter;

FIG. 5 shows adhesion efficiencies of six strains of novel Campylobacter and an invasive strain of C. jejuni (EX114);

FIG. 6 shows invasion efficiencies of six strains of novel Campylobacter and an invasive strain of C. jejuni (EX114);

FIG. 7 shows Campylobacter rMLST (Multi Locus Sequence Typing) based on 50 ribosomal allelic profiles;

FIG. 8 shows Campylobacter rMLST based on nucleotide sequences; and

FIG. 9 shows Campylobacter whole genome (wg) MLST.

### EXAMPLES

### Materials and methods

Cases and Collection of Specimens

Cases of SLS/SLD were investigated on nine free-range layer farms in England. The flocks had a fall in egg production and increased mortality with the typical gross lesions as described in the background section, above. The birds examined were untreated and were either freshly dead or culled for examination.

On all farms samples of affected liver were collected aseptically and macerated in Preston broth (Bolton et at (1983) J. Clin. Pathol. vol. 36 p 78-83; Hoffman et at (1979) Can. J. Microbiol. vol. 25 p 8-16). The broths were incubated for seven days at 37±2° C. Subcultures onto 5% sheep blood agar (Table 1) were incubated at 37±2° C. in a microaerobic atmosphere (2.5 litre AnaeroJar and CampyGen atmosphere generation system Oxoid Ltd Basingstoke). Plates were examined for evidence of growth after three and seven days of incubation. Growth was then sub-cultured for basic Campylobacter phenotypic confirmation.

On farms 3 and 4, in addition to the liver cultures, samples of bile were aspirated aseptically from the gall bladder. They were plated directly onto 5% sheep blood agar and incubated at 37±2° C. in a microaerobic atmosphere as above and examined after three and seven days of incubation. Growth was then sub-cultured for basic Campylobacter phenotypic confirmation.

Liver was fixed in 10 percent neutral phosphate-buffered formalin. After trimming to 3-5 mm thickness, the slices were processed to paraffin wax blocks. Sections were cut at 4 μm and stained with haematoxylin and eosin (H&E) and Warthin-Starry silver stain (Bancroft & Stevens (1996) Theory & Practice of Histological Techniques London, pub. Churchill Livingstone).

Basic Phenotypic Characterisation to Confirm Campylobacter spp.

The phenotypic characteristics of suspect isolates were determined using the minimal standards proposed for describing species of the family Campylobacteraceae (Ursing et al. (1994) Int. J. Syt. Bacteriol. vol. 44 p 842-845). Standardised phenotypic methodologies were used as described in ISO 10272-1:2006(E) (International Standard. Microbiology of food and animal feeding stuffs-Horizontal method for detection and enumeration of Campylobacter spp.: Part 1:Detection method, 2006). For phenotypic characterisation, suspect isolates were grown on 7% sheep blood agar plates with 0.1% cyclohexamide (SBA) (Table 1) and incubated at 37±2° C. and 41.5±1° C., in a microaerobic atmosphere (84% N₂, 10% CO₂, 6% O₂) for 48-96 hours before further testing. Cell morphology and motility were studied by microscopic examination. The ability to grow at various temperatures and under aerobic and microaerobic conditions was investigated. Basic phenotypic characterization included oxidase activity using commercially available sticks (Oxoid Ltd, Basingstoke); catalase activity using 3% hydrogen peroxide solution (Sigma-Aldrich Company Ltd, Gillingham) and observation of a positive reaction after 30 seconds; indoxyl acetate hydrolysis using indoxyl acetate discs (Sigma-Aldrich Company Ltd, Gillingham) and hippurate hydrolysis (1% sodium hippurate and 3.5% ninhydrin reagent in 1:1 butanol/ethanol).

Susceptibility to nalidixic acid (30 μg; Oxoid Ltd, Basingstoke) and cephalothin (30 μg; Oxoid Ltd, Basingstoke) was determined by disc diffusion method in Mueller-Hinton with 5% blood (Table 1) agar plates. A suspension of the bacteria in Brucella broth (Table 1) was prepared to McFarland Scale 0.5 and further diluted 1 in 9 in the same broth. Plates were incubated at 37° C.±2° C. for 22 h±2 h under microaerobic conditions as stated above. Growth that was in contact with the disc was classified as resistant and the presence of a zone of inhibition of any size was classified as susceptible.

Further Characterisation Studies

1. Bacterial Strains

A panel of isolates from suspect cases of spotty liver disease were investigated. Isolates were characterised, by both 16S sequence analysis and phenotypic tests, as the novel Campylobacter species. The reference culture strain deposited as ECACC

Deposit 12022102 (see below) was one of the 23 novel species strains, referred to as strain S12/1018.

2. Culture Media and Growth Conditions

For further phenotypic characterisation studies, all isolates were grown on SBA and incubated at 37° C. in a micro-aerobic atmosphere for 48-96 hours before testing. All tests were performed 37° C. in a micro-aerobic atmosphere unless stated otherwise.

3. Phenotypic Characterisation

A total of 30 phenotypic characteristics were determined for the novel Campylobacter strains. Further tests of growth in different incubation conditions and on different media types were performed using an inoculum of 1×10⁴ CFU/ml in Muller Hinton Broth (Table 1) and methods as recommended by On et al. (J. Clin. Micro, 1991, vol 25, pp 923-926). SBA was the plating medium used for testing growth in different incubation conditions and the presence of bacterial growth was assessed by eye after 96 hours incubation. Growth in micro-aerobic (84% N₂, 10% CO₂, 6% O₂) conditions were assessed at 25° C., 37° C., 41.5° C. Growth in aerobic and anaerobic conditions were assessed at 37° C. Growth in a micro-aerophilic atmosphere with hydrogen (87% N₂, 5% CO₂, 3% H₂, 3% Air) was assessed in VAIN incubator at 41.5° C.

Growth in the presence of 1% Glycine, 2% NaCl and 1% Bile were assessed by preparation of blood agar plates with 5% horse blood (HBA, Table 1) with the listed additional supplements. The plates were inoculated with 1×10⁴ CFU/ml in Muller Hinton Broth and the presence of growth assessed by eye after 96 hours micro-aerobic incubation at 37° C. Growth was also assessed on modified charcoal cefoperazone deoxycholate agar (MCCDA, Table 1).

The ability for reduction of selenite and tri-phenyl tetrazolium chloride (TTC) by the strains was assessed by inoculation onto HBA supplemented with 0.1% selenite and 0.04 TTC respectively. Plates were again examined for growth after 96 hours, orange growth on selenite plates and crimson growth on TTC plates indicated reduction. Anaerobic growth on HBA supplemented with 0.1% trimethylamine N-oxide (TMAO) was assessed after 96 hours incubation at 37° C. Nitrate reduction was by Cooks method as recommended by On et al. (J. Clin. Micro, 1992, vol 30, pp 746-749), HBA supplemented with 0.1% potassium nitrate was used. A loopful of sub-culture from SBA was inoculated onto a plate and micro-aerobically incubated at 37° C.; plates were checked after 24 and 48 hours incubation for dark zones around the inoculation point that indicates nitrate reduction.

The production of H₂S from growth on triple sugar iron agar (TSI) was assessed as recommended by Ursing et al. (Int. J. Syst. Bact, 1994, vol 44, pp 842-845), a loopful of sub-culture is stabbed into a tube of TSI agar (Table 1) and incubated at 37° C. for 48 hours in a hydrogen enriched atmosphere. Blackening at the site of growth indicates H₂S production. The production of H₂S in iron-bisulfite-pyruvate (FBP) agar (Table 1) was a described by Ursing et al. (1994), inoculation was a loopful and results read after 24-48 hours micro-aerobic incubation; black colouration indicates H₂S production. The production of H₂S from growth on sheep blood agar with 0.2% Cysteine (Table 1) was assessed following inoculation of 3×10⁷ CFU of sub-culture suspended in 50 μl of Mueller Hinton broth and then micro-aerobic incubation at 37° C. for 48-72 hours. Blackening of lead acetate paper that is attached above the growth/agar indicates H₂S production.

Glucose oxidation and fermentation were assessed by the Hugh and Leifson test (Ursing et al., 1994); a loopful of sub-culture was inoculated into 0/F media (Table 1) and incubation was at 37° C. micro-aerobic for 24-48 hours.

Urease production test was performed on a Christensens agar slope (Table 1) (Ursing et al., 1994); inoculation was a loopful of sub-culture and the results were read after 24-48 hours incubation as before, pink colouration indicating urease production.

The Alkaline phosphatase test was performed on a loopful of subculture as described by On et al. (1992) and Itoh et al. (Microbiol. Immunol, 1987, vol 31, pp 603-614). Results were read after 1 hour.

The γ-Glutamyltranspeptidase test was performed on a loopful of subculture as described by Feodoroff et al. (Gut. Pathogens, 2010, vol 2, pp 22).

All subcultures on SBA were examined by eye for the production of green tinged haemolysis zones surrounding colonies; these are indicative of α-haemolysis after 48 and 72 hours growth.

4. Antimicrobial Resistance Testing

Antimicrobial susceptibility testing was performed using the agar dilution method for the Campylobacter novel strains against 14 antimicrobials. The test was performed following the British Society for Antimicrobial Chemotherapy (BSAC) guidelines (B SAC Methods for Antimicrobial Susceptibility Testing, Version 10.2, May 2011, Birmingham, United Kingdom). As the strains were unable to grow on the Iso-sensitest agar (ISA) recommended by the BSAC guidelines, HBA (Table 1) was used as an alternative media.

5. Matrix Assisted Laser Desorption Ionization Time of Flight (MALDI-TOF) Identification

Defined reference protein spectra were obtained for all the strains included in the study (main spectral projections (MSP) using MALDI Biotyper 2.0 software, Bruker Daltonics, Bremen, Germany). The MSP represent individual protein spectra for one bacterial strain. To achieve a representative MSP, at least 20 individual spectra of each novel strain were used to create a single MSP for that strain as proposed.

Individual reference spectra were generated by testing ethanol/formic acid extracts of each strain by MALDI-TOF. Extractions of fresh sub-cultures from SBA (72 hours micro-aerobic incubation at 37° C.) were made as described in the MALDI Biotyper 2.0 user manual (Version 2.0 SR1, October 2008, Chapter 3), and then stored at −20° C. until further processing by MALDI-TOF. Individual spectra were generated from the extracts using an Auto-Flex 2, (Bruker, Bremen, Germany) following conditions described in the MALDI Biotyper user manual (Chapter 3). Individual spectra were screened for quality and at least 20 suitable spectra were collected for the creation of the MSP (MALDI Biotyper user manual, Chapter 8 and 9). The MSP were created automatically by the software and all the new MSP were added to the main spectra library as unassigned MSPs. Using the software MALDI Biotyper™ all 26 (one per strain) MSP spectra were visualized in a dendrogram (MALDI Biotyper user manual, Chapter 14).

The spectra from each strain were also compared with the Bruker Campylobacter and Helicobacter reference library using a score value, using a common decadal logarithm for matching results. Results were analyzed following the score value system as described in the MALDI Biotyper user manual (Chapter 15).

6. Electron Microscopy

Five strains of the novel Campylobacter (designated S10/0209, S11/0069, S11/0071, S12/002 and S12/1018) were grown for 72 hrs at 42° C. in a microaerobic environment. S12/1018 is the strain which was deposited as ECACC Deposit 12022102 (see below). Cultures were harvested and washed once in phosphate buffered saline (PBS) and centrifuged at 2100×g for 15 minutes to pellet the cells. Bacterial pellets were re-suspended in 100-500 mL PBS. Droplets of 50 ml of each bacterial suspension were placed on a sheet of dental wax (Kemdent®). Formvar™ carbon coated EM grids were floated on the bacterial suspension for five minutes. Grids were then blotted dry and placed onto a drop of 2% (v/v) phosphotungstic acid (pH 6.6) negative stain for 10 seconds. Grids were carefully blotted dry and viewed using a Philips CM 10 transmission electron microscope.

7. In Vitro Invasion and Adhesion Assays

The adhesion and invasion potentials of all strains were determined using in vitro invasion and adhesion assays as described (Javed M A, et al. (2010) Microbiol. vol. 156 pp 1134-1143). Briefly, six strains of the novel Campylobacter (designated S10/0209, S11/013, S11/0069, S11/0071, S12/002 and S12/1018) were cultured for 72 hrs in brain-heart infusion broth/agar biphasic flasks. In addition a C. jejuni strain of known high invasive potential (EX114) was included as a control. Bacteria were harvested by centrifugation, washed×1 in PBS and resuspended in PBS to give an OD550 of about 0.75, equivalent to approximately 10⁸ cfu/ml.

Monolayers of INT-407 cells were grown in two 24-well cell culture plates. The monolayers were covered with bacterial cells at a multiplicity of infection of 100 in complete cell culture medium and incubated at 37° C. for 3 h in 5% (v/v) CO₂. The monolayers were washed 3 times with PBS. To determine adhesion, the cells from one plate were lysed by adding 1 ml 1% (v/v) Triton X-100, and bacterial numbers determined by quantitative bacteriology.

To determine invasiveness, the other plate was incubated for a further 2 h with 1 ml of complete cell culture medium supplemented with 250 mg/ml gentamicin in each well. Following incubation, the monolayers were washed three times with PBS and lysed as above.

Assays were performed in triplicate. The numbers of adherent bacteria were calculated by subtracting the number of internalized bacteria from the total number counted. Invasion/adhesion efficiencies were expressed as the percentage of the inoculum that adhered/invaded.

TABLE 1
Media ingredients for the phenotypic testing

| Media | Recipe |
|---|---|
| 5% Sheep | Columbia blood agar base (Oxoid CM0331 or |
| blood agar | equivalent) - 39 g |
| | Distilled/deionised water - 950 ml |
| | Sterile defibrinated sheep's blood - 50 ml |
| 7% Sheep blood | 40 g Blood agar base (Oxoid CM0055) |
| agar with 0.1% | 70 ml sterile defibrinated sheep blood, 1000 mg |
| cyclohexamide | cyclohexamide, 1000 ml sterile |
| (SBA) | distilled/deionised water |
| Mueller Hinton | Columbia blood agar base (Oxoid) - 38 g |
| Agar with | Distilled/deionised water - 950 ml |
| 5% blood | Sterile defibrinated sheep's blood - 50 ml |
| Brucella | Tryptone Soya Broth (Oxoid) 30 g |
| Broth | Distilled Water 1000 ml |
| | Heated Horse Serum 50 ml |
| | Bactitracin (2000 iu/ml) 10 ml |
| | Polymixin B (1000 iu/ml) 5.0 ml |
| | 0.01% Cycloheximide (100 ng/ml) 5.0 ml |
| | 5% Naladixic Acid (0.05 g/ml) 1.0 ml |
| | 1% Vancomycin (10 mg/ml) 2.0 ml |
| | Nystatin (5 × 104 iu/ml) 20 ml |
| | 0.5% Fungizone (5 mg/ml) 0.8 ml |
| Mueller | Mueller Hinton Broth (Oxoid) 21.0 g |
| Hinton Broth | Distilled Water 1000 ml |
| 5% Horse | Blood Agar Base [BAB] (Oxoid) 40 g |
| blood agar | Distilled Water 1000 ml Sterile defibrinated |
| (HBA) | horse blood - 50 ml |
| Triple Sugar | Triple Sugar Iron Agar (Oxoid) 65.0 g |
| Iron agar | Distilled Water 1000 ml |
| (TSI) | |
| Modified charcoal | 45.5 g Campylobacter blood free selective |
| cefoperazone | agar base (Oxoid, CM0739). |
| deoxycholate | 32 mg cefoperazone, |
| agar (MCCDA) | 16 mg amphotericin |
| | Distilled Water 1000 ml |
| Iron bi-sulphate | Nutrient Broth No2 (Oxoid) 25 g |
| pyruvate | Agar 1.2 g |
| (FBP) agar | Yeast Extract 1.0 g |
| | Campylobacter Growth Supplement SR0232 |
| | (Oxoid) (4 vials) 8 ml |
| | Glycerol 150 ml |
| | Distilled Water 850 ml |
| Sheep blood | Oxoid Blood Agar Base No. 2 (CM0271) or |
| agar with | equivalent - 20 g |
| 0.2% cysteine | 0.02% Cysteine VWR 23257.183 or |
| | equivalent - 10 ml |
| | Distilled/De-ionised water - 465 ml |
| | Sterile defibrinated Sheep blood - 25 ml |
| Glucose | Difco OF Basal Medium ref 268820 (or |
| oxidation/fermenta- | equivalent) - 9.4 g |
| tion media (O/F) | Distilled/deionised water -950 ml |
| | 20% sterileglucose solution - 50 ml |
| Christensens agar | 2.4 g Urea Agar base (Oxoid CM53) |
| | 5 ml of 40% urea solution (Oxoid SR20) |
| | 95 ml Deionized/Distilled water |

Sequence Analysis

1. 16S rRNA Sequencing

Sequencing of the 16S rRNA gene was carried out to provide preliminary molecular confirmation of the identity of the bacterial isolates and to place them relative to currently described species. In brief, crude lysates were prepared from a pure culture by adding a suitable small colony into 100 μL of molecular grade water and heating for 10 min at 98° C. Using this lysate as DNA template the virtually complete 16S rRNA gene was amplified from strains by PCR using primer pair:

(SEQ ID NO: 3)
5′-AGTTTGATCCTGGCTCAG-3′
(SEQ ID NO: 4)
5′-ACGGTACCTTGTTACGACTT-3′

The resulting PCR products were sequenced using a series of internal primers. Phylogenetic analysis was carried out using MEGA5 (Tamura et al. (2011) Mol. Biol. Evol. Vol. 28 p 2731-2739) in comparison with sequences of type strains identified through the “List of Prokaryotic names with Standing in Nomenclature” at http://www.bacterio.cict.fr/ and downloaded from GenBank.

2. Whole Genome Sequencing; Ribosomal and Whole Genome MLST (Multi Locus Sequence Typing) Analysis

Ten strains of novel Campylobacter associated with the spotty liver disease were chosen for analysis using Whole Genome Sequencing (WGS). The chosen strains with their Campylobacter lab reference and DNA concentration as measured using Qubit instrument (Invitrogen) are listed in Table 2.

All strains were stored at −80° C. in 1% (w/v) proteose peptone water containing 10% (v/v) glycerol until required. Strains were grown from frozen on 10% (v/v) sheep blood agar plates containing Skirrow's antibiotics (Vancomycin (10 mg/mL), Polymyxin B (2.5 i.u./mL) Trimethoprim (5 mg/mL) and Actidione (250 mg/mL) (BASA); (Skirrow, 1977, Br. Med. J. vol 2 pp 9-11)) at 42° C. in a microaerobic atmosphere (85% (v/v) N₂, 7.5% (v/v) CO₂, 7.5% (v/v) O₂). The bacteria from each plate were harvested into tubes containing 1 mL 0.1 M PBS (pH 7.2) solution. Cells were pelleted by centrifugation and re-suspended in 0.5 ml 0.1M PBS (pH7.2) solution. DNA was extracted using the ArchivePure DNA Cell/Tissue (1 g) kit (5Prime, Gaithersburg, USA) following manufacturer's guidelines.

TABLE 2
sequenced strains

| strain lab reference | DNA conc μg/ml |
|---|---|
| S10/0209 | 368 |
| S11/0010 | 315 |
| S11/0013 | 72.5 |
| S11/0036 | 52.1 |
| S11/0038 | 67.5 |
| S11/0069 | 165 |
| S11/0071 | 97.9 |
| S12/0002 | 137 |
| S12/0322 | 102 |
| S12/1018 | 172 |

Genomic DNA was fragmented and tagged for multiplexing with Nextera XT DNA Sample Preparation Kits (Illumina) and sequenced using the AHVLA Illumina MiSeq instrument using 150 bp paired end reads.

Assembled data were deposited on the pubmlst.org/campylobacter database. A total of 1667 loci are defined in this database, including the MLST loci, ribosomal 99 MLST (rMLST) loci, porA antigen-encoding genes, and other loci derived from 100 sources, including the re-annotation of the sequence of Campylobacter jejuni isolate 11168. The database comparison was run at Oxford University (Melisa Jansen van Rensburg). Ribosomal MLST (rMLST) comparison was performed using 50 ribosomal allelic profiles and also nucleotide sequence data. Whole genome MLST (wgMLST) was also performed to improve genus resolution.

Results

The Outbreaks

There was an increased mortality with typical pathology on all nine farms. The five outbreaks involving four farms where the novel Campylobacter was originally isolated are described below. An isolate was also obtained from a fifth farm, also described below. No isolates were made when investigating the other four farms.

Farm 1 Devon—This eight house, multi-age site suffered two outbreaks; one in September 2009 and a second in June 2011.

In September 2009 there was increased mortality in a house of 6,000, 27-week-old layers. Mortality at the time of submission had reached 1.7%. In June 2011 a second outbreak was investigated in a house of 7,000, 20-week-old layers. They never reached peak production and throughout the life of the flock production was between 15 and 35% below target. At the time of submission mortality was 2.2%.

Farm 2 Essex—There was a sudden increase in mortality in a flock of 5,500 40-week-old free-range layers in August 2011. Mortality peaked at 40 weeks with 50 losses. Prompt antimicrobial treatment appeared to limit the mortality with only 20 birds lost in the following week. Egg production fell by 40%, though there was also a concurrent change in feed. Loose faeces were noted in the flock. Heterakis species worms were present in the caecae.

Farm 3 Devon—In September 2011 there was an increased mortality in one house of 5,000, 26-week-old layers on a multi-age site.

Farm 4 Cleveland—Mortality occurred in a single 11,300 bird house. The birds were 44 weeks old at onset of mortality and 70-90 birds were lost each day. There was a fall in egg production of 10%. Ascaridia species and Heterakis species worm eggs were noted on post-mortem examination.

Farm 5 Lincolnshire—A fall in egg production of 5-6% occurred in two out of eight houses of 47 week old layers. There were 63,000 birds on the multiage site.

Pathology

Post-mortem examination of birds from each affected farm revealed characteristic multifocal 1-2 mm grey-white lesions in the liver. A proportion of birds also had a fibrinous perihepatitis and excess, clear abdominal and/or pericardial fluid. There was pallor of the kidneys in some birds. In all cases liver histopathology showed acute multifocal necrosis and fibrinogranulocytic hepatitis, with multiple random foci of necrosis containing small amounts of fibrinous exudate. There was often a variable though usually light, mixed heterophil and macrophage infiltration. Bacteria were not seen within the lesions with either H&E or Warthin-Starry stains.

Bacteriology & Initial Characterisation

Isolates were made from the six outbreaks (Table 3). Eighteen of the isolates were examined for their phenotypic characteristics and twenty by 16S rRNA sequencing.

TABLE 3
Cultures and isolates of the novel Campylobacter

| | Cultures | | Isolates | |
|---|---|---|---|---|
| | Liver | Bile | Liver | Bile |
| Farm 1 2009 | 2 | 0 | 2 | 0 |
| Farm 1 2011 | 18 | 0 | 7 | 0 |
| Farm 2 2011 | 4 | 0 | 4 | 0 |
| Farm 3 2011 | 4 | 8 | 0 | 3 |
| Farm 4 2011 | 6 | 4 | 4 | 2 |
| Farm 5 2012 | 5 | 0 | 2 | 0 |
| Total | 39 | 12 | 19 | 5 |

On 5% sheep blood agar there was slow, variable, uneven growth. Visible growth usually first appeared at three days incubation though colonies sometimes took up to seven days to appear on first isolation. The presence of any faster growing contaminant bacteria precluded the isolation of the novel Campylobacter.

Growth took the form of a thin spreading film on the agar surface or round dirty yellow watery colonies of variable size. If the initial growth was a film, then colonies of varying size often appeared on the spreading film after further incubation. There was a very weak alpha-haemolysis. Gram stain on initial isolation showed Gram negative curving rods but on passage, Gram stain showed nearly exclusively Gram negative coccoid forms.

The isolates were predominantly highly motile in young cultures but were less actively motile in more mature cultures. The organisms grew under microaerobic conditions at 37° C. and most isolates also grew at 41.5° C. None of the isolates grew at 25° C. either microaerobically or aerobically. Isolates were typically oxidase and catalase positive. The isolates had either negative or very weak hydrolytic activity for both indoxyl acetate or sodium hippurate, that is typically considered negative in standard phenotypic tests. The isolate had variable resistance to cephalothin (Table 4).

Further Characterisation Results

1. Phenotypic Description of Novel Campylobacter from Suspect Spotty Liver Disease (n=23)

Results of the further phenotypic characterisation of the novel Campylobacter strains isolated from suspect cases of spotty liver disease are presented in Table 5. Cells are gram-negative rods and motile. On sheep blood agar at 37° C. under microaerobic conditions, colonies appeared α-haemolytic, fine shiny greyish colonies with areas of heavy growth smooth and shiny. Some strains exhibited larger swarming shiny greyish colonies. Most strains showed a slow or very slow growth with 3-4 days required for good growth to appear on the plates.

Strains were strictly microaerobic, able to grow at 37° C. and 41.5° C., but not at 25° C. or under anaerobic and aerobic conditions and did not show a particular requirement for hydrogen to grow. Oxidase and very strong catalase activity were observed, but not urease production. All strains exhibited γ-glutamyltranspeptidase and alkaline phosphatase activity. All strains tested negative for indoxyl acetate hydrolysis and had no or very weak activity for hippurate hydrolysis, that is considered hippurate test negative, relative to a positive control strain (C. jejuni). They did reduce nitrate but not selenite. Strains were variable in the ability to reduce triphenyl tetrazolium chloride. Strains did not produce H₂S in TSI agar but produced H₂S in cysteine agar. All strains grew in the presence of 1% bile and most can grow in the presence of 1% glycine. No growth occurred in the presence of 2% NaCl. Growth on mCCDA appears after 3-4 days of incubation although growth on this medium was restricted for some strains. Strains were susceptible to nalidixic acid (30 μg) and showed variable resistance to cephalothin (30 μs) by disc diffusion tests.

There were phenotypic characteristics exhibited by the novel Campylobacter strains that differentiate them from the typical C. jejuni strains (C. jejuni jejuni and C. jejuni doylei reference strains): they had either no or very weak activity for hippurate hydrolysis. They tested negative for indoxyl acetate hydrolysis but they exhibited γ-glutamyltranspeptidase and alkaline phosphatase activity. For some novel Campylobacter strains growth on mCCDA was restrictive which is different from both subspecies of C. jejuni and from typical C. coli strains. The novel Campylobacter strains were similar to C. jejuni subsp. doylei, in that they did not reduce selenite, some did not reduce triphenyl tetrazolium chloride, some did not grow in presence of 1% glycine and some were sensitive to cephalothin.

In Table 5, taxa are as follows:

| A: | Campylobacter novel |
|---|---|
| 1: | C. avium sp. Nov |
| 2: | C. canadensis |
| 3: | C. coli |
| 4: | C. concisus |
| 5: | C. cuniculurom |
| 6: | C. curvus |
| 7: | C. fetus subsp. fetus |
| 8: | C. fetus subsp. venerealis |
| 9: | C. gracilis |
| 10: | C. helveticus |
| 11: | C. hominus |
| 12: | C. hyointestinalis subsp. hyointestinalis |
| 13: | C. hyointestinalis subsp. lawsonii |
| 14: | C. insulaenigrae |
| 15: | C. jejuni subsp. Doylei |
| 16: | C. jejuni subsp. Jejuni |
| 17: | C. lanienae |
| 18: | C. lari |
| 19: | C. mucosalis |
| 20: | C. rectus |
| 21: | C. showae |
| 22: | C. sputorum |
| 23: | C. upsaliensis |

Data for reference species were taken from On et al. (Clinical Microbiology reviews, 1996, vol 9, 405-422), Foster et al. (Int. J. Syst. Evol. Microbiol, 2004, vol 54, pp 2369-2373), Vandamme et al. (Int. J. Syst. Evol. Microbiol, 2010, vol 60, pp 2016-2022) Bergeys Manual of Systematic Bacteriology (2005, 2^{nd} Ed, volume 2, Part C, pp 1145-1164, Springer, New York, USA), Inglis et al. (Int. J. Syst. Evol. Microbiol, 2007, vol 57, pp 2636-2644) and Zanoni et al. (Int. J. Syst. Evol. Microbiol, 2009, vol 59, pp 1666-1671), Rossi et al., (Int. J. Syst. Evol. Microbiol, 2009, vol 59, pp 2364-2369), Bergeys Manual of Determinative Bacteriology (1994, 9^{th} Ed, Group 2, pp 39-62, Williams and Wilkins, Baltimore, USA), Topley and Wilsons Microbiology and Microbial Infections (9th Ed, 1998, Volume 2, Systematic Bacteriology, Chap 54, pp 1239, Arnold, London).

All taxa were negative for aerobic growth at 37° C.

2. MALDI Identification

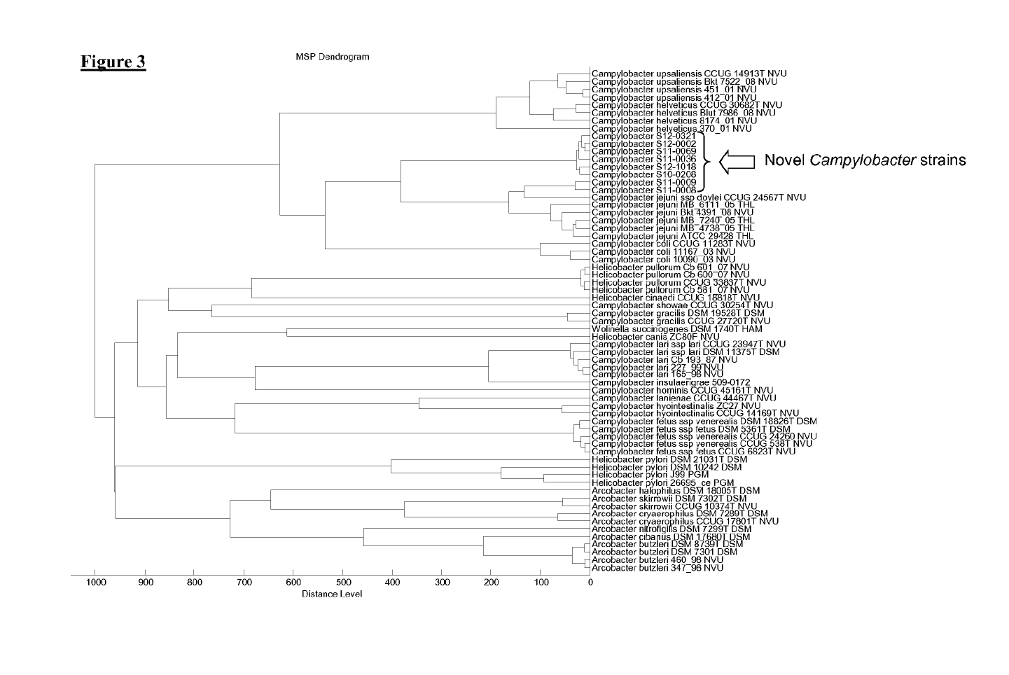

As described above, defined reference protein spectra were obtained for all the strains included in the study (main spectral projections (MSP)). Individual reference spectra were generated by testing formic acid extracts of each strain by the MALDI. Individual spectra were screened for quality and at least 20 suitable spectra were collected for the creation of the MSP. The MSP were created automatically by the software and all the new MSP were added to the main spectra library as unassigned MSPs. Using the MALDI Biotyper 2.0™ software all 26 (one per strain) MSP spectra were visualized in a dendrogram (FIG. 3) along with other species of Campylobacter and related members of the Family. The strains of the novel species could clearly be differentiated from the other Campylobacter species and related taxa. It was interesting to note that the MSP for the spotty liver C. jejuni were clustered with C. jejuni species but closer to the C. jejuni doylei subspecies.

The spectra from each strain were also compared with the Bruker Campylobacter and Helicobacter reference library using a score value, using a common decadal logarithm for matching results. Results were analysed following the score value system, as outlined above. The novel Campylobacter species were all identified as the novel species with a score of value of greater than 2.3, indicating a highly probable species identification. A score of >2 is required for secure genus identification and probable species identification according to Bruker. None of the novel Campylobacter strains matched with C. jejuni with a score greater than 2.0.

3. Antimicrobial Resistance Testing

The distribution of MIC, MIC₅₀ and MIC₉₀ values of the novel Campylobacter strains is shown in Table 6.

Strains showed low MICs against: Ampicillin (MIC₅₀=1 μg/ml and MIC₉₀=2 μg/ml), Ciprofloxacine (MIC₅₀=0.06 μg/ml and MIC₉₀=0.06 μg/ml), Streptomycin (MIC₅₀=1 μg/ml and MIC₉₀=1 μg/ml), Erythromycin (MIC₅₀=1 μg/ml and MIC₉₀=2 μg/ml), Gentamycin (MIC₅₀=0.5 μg/ml and MIC₉₀=0.5 μg/ml) and Tiamuline (MIC₅₀=2 μs/ml and MIC₉₀=2 μg/ml).

Strains showed medium MICs against: Chloramphenicol (MIC₅₀=4 μg/ml and MIC₉₀=8 μg/ml), Kanamycin (MIC₅₀=4 μg/ml and MIC₉₀=4 μg/ml) and Nalidixic acid (MIC₅₀=4 μg/ml and MIC₉₀=8 μg/ml).

High MIC values were observed for Cefoperazone (MIC₅₀=64 μg/ml and MIC₉₀=64 μg/ml) and cephalothin (MIC₅₀=32 μg/ml and MIC₉₀=32 μg/ml).

A wider range of MIC values was observed for Colistin (MIC₅₀=8 μg/ml and MIC₉₀=32 μg/ml), Polymyxin B (MIC₅₀=8 μg/ml and MIC₉₀=16 μs/ml) and Tetracycline (MIC₅₀=1 μg/ml and MIC₉₀=64 μg/ml).

4. Electron Microscopy

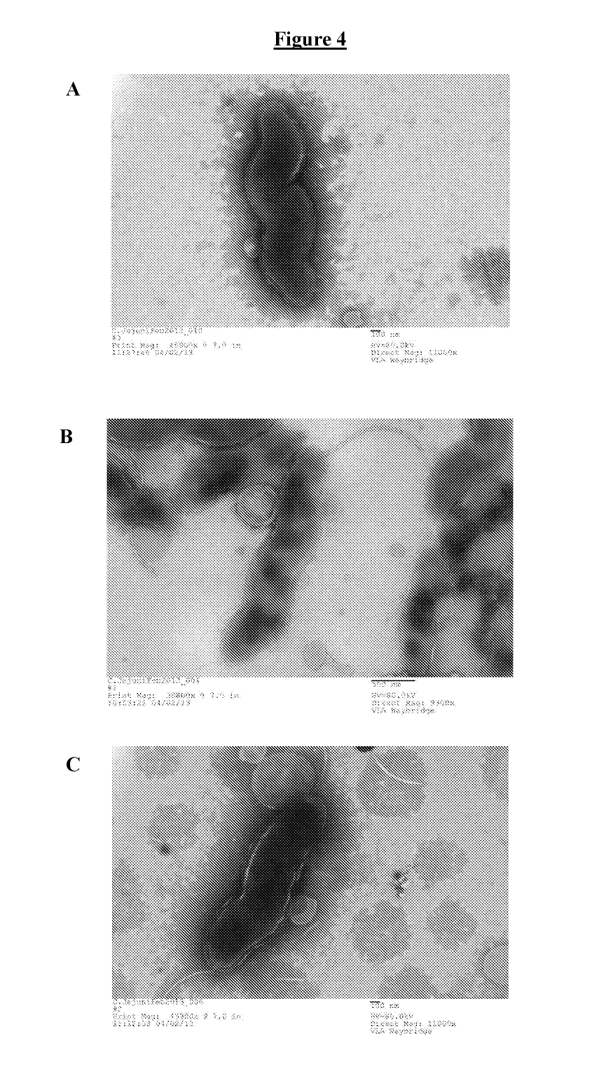

Electron micrographs (FIG. 4A for strain S11/0071, FIG. 4B for strain S10/209) and FIG. 4C for strain S11/069) revealed all strains showed typical Campylobacter morphology: curved rods approximately 0.5 μm in diameter and 2-3 μm in length. Bacteria had single, unipolar or bipolar, unsheathed flagella, although two strains (S11/0071 and S12/1018) appeared aflagellate.

5. In Vitro Invasion and Adhesion Assays

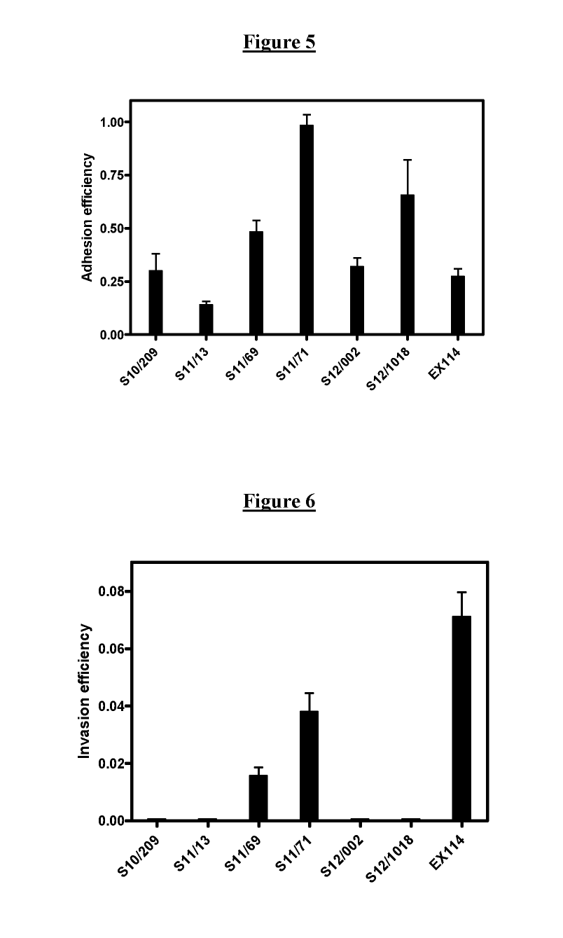

As shown in FIG. 5, there was some variability between the strains of novel Campylobacter in the ability to adhere, but efficiencies were similar or greater than that of the invasive C. jejuni strain EX114. FIG. 6 shows that, in contrast, the novel campylobacters were poor invaders in comparison to the C. jejuni strain. Indeed, four of the strains only just reached the limit of detection. Strains S11/69 and S11/71 were considerably more invasive than the other strains. Similar variations in invasive potential have been observed previously in C. jejuni strains (Fearnley et al. (2008) J. Med. Microbiol. vol. 57 pp 570-80).

Sequencing Analysis

1. 16S rRNA Sequencing

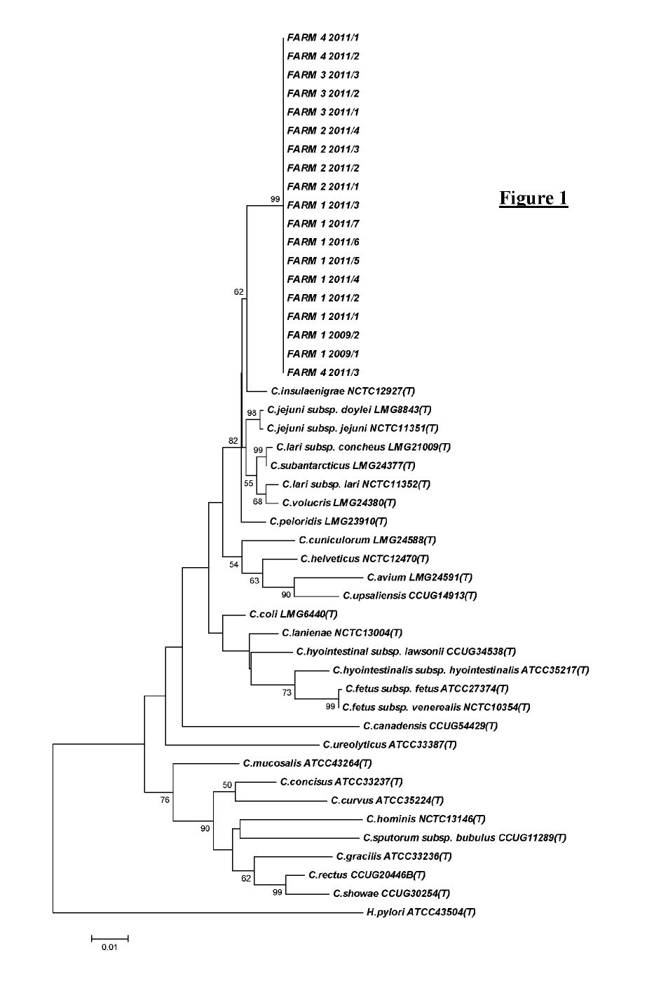

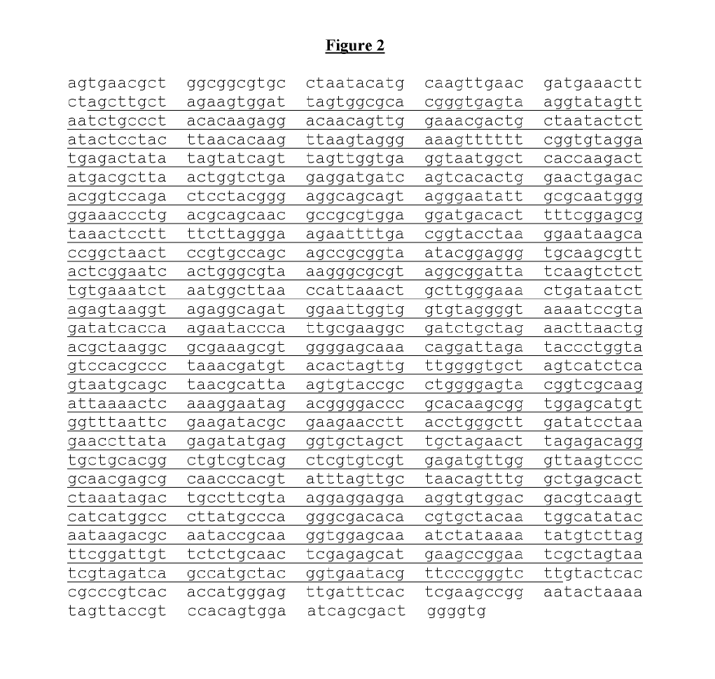

Sequencing of 16S rRNA revealed that the 19 bacterial isolates cultured from farms 1-4 shared identical sequence over a common 1290 bp fragment (SEQ ID NO:1), which forms part of the longer sequence SEQ ID NO:2, 1436 bp. The sequence from an isolate from Farm 5 is SEQ ID NO:5, which differs from SEQ ID NO:1 in a G→A substitution at position 1141 of SEQ ID NO:1. Phylogenetic analysis (illustrated in FIG. 1) and comparison of sequences with equivalent regions of the type strains of all currently described Campylobacter species, revealed that the isolates appeared to represent a hitherto unknown sublineage within the group. The isolates from Farm 5 also clustered to this sublineage. Pairwise sequence comparisons over the same 1290 bp sequence revealed highest similarities with type strains of C. jejuni subsp. jejuni (98.45%), C. jejuni subsp. doylei (98.45%), C. subantarcticus (98.37%), C. insulaenigrae (98.37%) and C. lari subsp. concheus (98.21%). Levels of similarity with other Campylobacter species, such as C. coli (97.21%) were substantially lower. SEQ ID NO:5 is over 99.9% identical to SEQ ID NO:1.

An isolate of the bacterium has been deposited as Deposit reference 12022102 (“Campylobacter species 21/B124/06”) with European Collection of Cell Cultures, Porton Down, United Kingdom, in accordance with the Budapest Treaty on the International Recognition of the Deposit of Micro-organisms for the Purposes of Patent Procedure. The deposit was made by Dr Timothy Crawshaw, address Animal Health & Veterinary Laboratories, Staplake Mount, Starcross, Exeter, Devon EX6 8PE, United Kingdom, on 21 Feb. 2012.

2. Whole Genome Sequencing; Ribosomal and Whole Genome MLST Analysis

Ten genome sequences from the strains set out in Table 2 above were assembled de novo using VELVET 94 version 1.2.01 shuffle and optimisation scripts to create contigs using optimal parameters, with 95 kmer lengths between 49 and 69 bp (Zerbino, 2010, Curr. Protoc. Bioinformatics Chapter 11, Unit 11.5; doi: 10.1002/0471250953.bi1105s31). Genomes were annotated using Prokka annotation pipeline and aligned using MAUVE genome aligner for genome comparison.

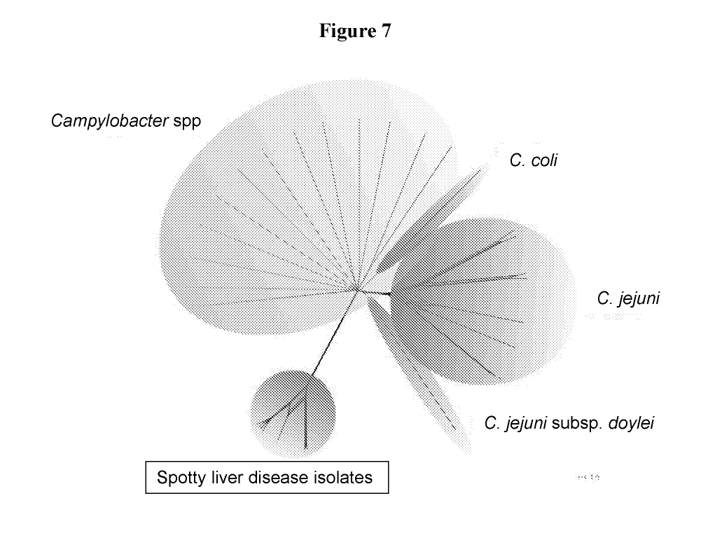

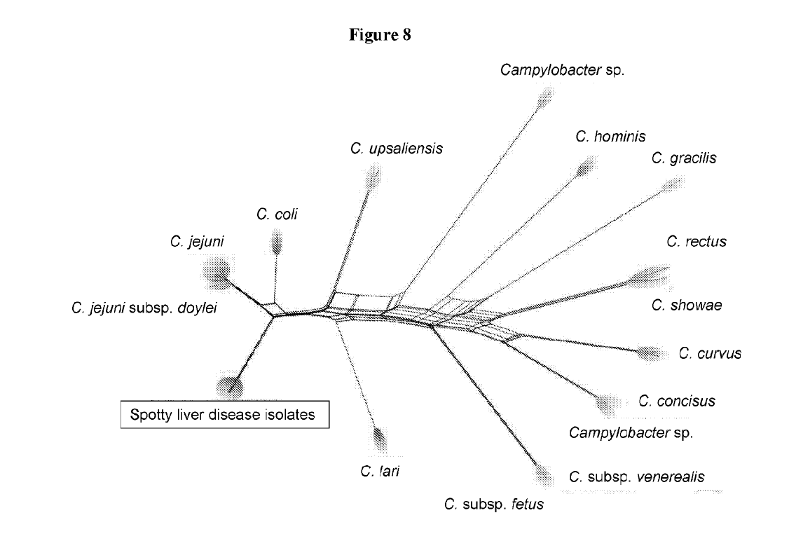

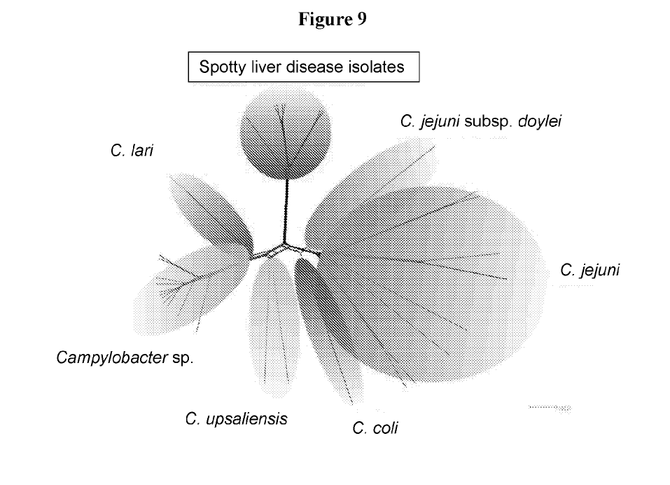

As described above, Ribosomal MLST (rMLST) comparison was performed using 50 ribosomal allelic profiles (FIG. 7) and also nucleotide sequence data (FIG. 8). Analysis based on the allelic profiles grouped the isolates as shown in FIG. 7. Whole genome MLST (wgMLST) was also performed to improve genus resolution. The output (FIG. 9) is based on allelic profiles, rather than nucleotide sequence. Based on this analysis, the spotty liver disease isolates form a distinct group closest to C. jejuni and C. coli, as well as C. upsaliensis and C. lari.

TABLE 4
Phenotypic characteristics of novel campylobacter isolates recovered from the livers of free-range layer hens

| | | | | | | | | Susceptibility to | |
|---|---|---|---|---|---|---|---|---|---|
| | | Hydrolysis of: | | | Microaerobic | | Aerobic | (30 μg disc): | |
| | Oxidase | | Indoxyl | | growth | | growth | Nalidixic | |
| Strain Ref | activity | Catalase | acetate | Hippurate | 25° C. | 41.5° C. | 25° C. | acid | Cephalothin |
| Farm 1 2009/1 | + | + | +^{w} | − | − | + | − | S | R |
| Farm 1 2009/2 | + | + | +^{w} | − | − | + | − | S | R |
| Farm 1 2011/1 | + | + | − | − | − | + | − | S | R |
| Farm 1 2011/2 | + | + | − | − | − | + | − | S | R |
| Farm 1 2011/3 | + | + | − | − | − | + | − | S | R |
| Farm 1 2011/4 | + | + | +^{w} | − | − | + | − | S | R |
| Farm 1 2011/5 | + | + | +^{w} | − | − | + | − | S | R |
| Farm 1 2011/6 | + | + | − | − | − | + | − | S | R |
| Farm 2 2011/1 | + | + | − | − | − | + | − | S | S |
| Farm 2 2011/2 | + | + | − | − | − | + | − | S | S |
| Farm 2 2011/3 | + | + | − | +^{w} | − | + | − | S | S |
| Farm 2 2011/4 | + | + | − | − | − | + | − | S | S |
| Farm 3 2011/1 | + | + | +^{w} | + | − | + | − | S | S |
| Farm 3 2011/2 | + | + | +^{w} | + | − | + | − | S | S |
| Farm 3 2011/3 | + | + | +^{w} | + | − | + | − | S | S |
| Farm 4 2011/1 | + | + | +^{w} | − | − | − | − | S | S |
| Farm 4 2011/2 | + | + | +^{w} | − | − | − | − | S | S |
| Farm 4 2011/3 | + | + | − | − | − | − | − | S | S |

+, positive reaction;
−, negative reaction;
+^{w}, weak positive reaction;
S, susceptible;
R, resistant.

TABLE 5
Phenotypic characteristics of Campylobacter species

| | Taxa | | | | | | |
|---|---|---|---|---|---|---|---|
| Characteristic | A | 1 | 2 | 3 | 4 | 5 | 6 |
| α-haemolysis | + | − | − | (−) | (−) | + | (−) |
| Oxidase | + | + | + | + | V | + | + |
| Catalase | + | W | V | + | − | + | − |
| Alkaline phosphatase | + | − | − | − | V | − | V |
| γ-Glutamyltranspeptidase | + | − | (+) | − | − | − | NA |
| Urease production | − | − | V | − | − | − | − |
| Hippurate hydrolysis | − | + | − | − | − | − | (−) |
| | (w) | | | | | | |
| Indoxy acetate hydrolysis | − | + | − | + | − | + | V |
| Nitrate reduction | + | + | V | + | (−) | + | + |
| Selenite reduction | − | − | NA | V | (−) | − | − |
| TTC reduction | V | − | NA | + | − | V | V |
| H₂S on TSI agar | − | − | V | − | − | − | (−) |
| Growth 25° C. microaerobic | − | − | − | − | − | − | − |
| Growth 37° C. microaerobic | + | + | + | + | + | + | V |
| Growth 41.5° C. microaerobic | + | + | + | + | (+) | (+) | V |
| Growth 37° C. anaerobic | − | − | + | − | + | − | + |
| MCCDA | V | − | + | + | (−) | (+) | (+) |
| 1% Glycine | (+) | − | V | + | (−) | − | + |
| 2% NaCl | − | − | NA | − | (−) | − | V |
| 1% Bile | + | V | NA | (+) | − | V | − |
| Requirement for H2 | − | V | − | − | + | − | + |
| Nalidixic acid resistance | − | − | V | − | (+) | V | + |
| Cephalothin resistance | V | + | − | + | − | (+) | − |
| H₂S on Cysteine agar\hat{ }\hat{ }\hat{ }\hat{ } | + | NA | NA | NA | + | NA | NA |
| Anaerobic growth on TMAO | − | NA | NA | − | − | NA | NA |
| Aerobic growth | − | NA | NA | − | − | NA | NA |
| Glucose oxidation | − | − | − | − | − | − | − |
| Glucose fermentation | − | − | − | − | − | − | − |
| H₂S in FPB medium | − | NA | NA | − | − | NA | NA |

| | Taxa | | | | | | | |
|---|---|---|---|---|---|---|---|---|
| Characteristic | 7 | 8 | 9 | 10 | 11 | 12 | 13 | 14 |
| α-haemolysis | − | V | − | + | NA | V | V | NA |
| Oxidase | + | + | − | + | + | + | + | + |
| Catalase | + | (+) | V | − | − | + | + | + |
| Alkaline phosphatase | − | − | − | − | − | − | (−) | NA |
| γ-Glutamyltranspeptidase | − | NA | NA | − | NA | − | − | NA |
| Urease production | − | − | − | − | − | − | − | − |
| Hippurate hydrolysis | − | − | − | − | − | − | − | − |
| Indoxy acetate hydrolysis | − | − | V | + | − | − | − | − |
| Nitrate reduction | + | + | (+) | + | − | + | + | + |
| Selenite reduction | (+) | − | − | − | − | + | + | NA |
| TTC reduction | − | − | − | − | NA | − | − | NA |
| H₂S on TSI agar | − | − | − | − | − | + | + | − |
| Growth 25° C. microaerobic | + | + | − | − | − | − | − | − |
| Growth 37° C. microaerobic | + | + | + | + | + | + | + | + |
| Growth 41.5° C. microaerobic | (+) | − | V | + | (−) | + | + | − |
| Growth 37° C. anaerobic | (−) | V | + | − | + | − | + | − |
| MCCDA | + | + | V | + | NA | + | + | NA |
| 1% Glycine | + | − | + | V | + | + | V | + |
| 2% NaCl | − | − | V | − | + | − | − | − |
| 1% Bile | + | + | − | + | V** | + | (+) | NA |
| Requirement for H2 | − | − | + | − | + | V | V | NA |
| Nalidixic acid resistance | + | V | V | − | V | + | + | + |
| Cephalothin resistance | − | − | − | − | − | (−) | − | + |
| H₂S on Cysteine agar\hat{ }\hat{ }\hat{ }\hat{ } | + | − | NA | NA | NA | + | + | NA |
| Anaerobic growth on TMAO | − | − | NA | NA | NA | + | NA | NA |
| Aerobic growth | − | − | NA | NA | NA | − | NA | NA |
| Glucose oxidation | − | − | − | − | − | − | − | − |
| Glucose fermentation | − | − | − | − | − | − | − | − |
| H₂S in FPB medium | − | − | NA | − | NA | NA | NA | NA |

| | Taxa | | | | | | | | |
|---|---|---|---|---|---|---|---|---|---|
| Characteristic | 15 | 16 | 17 | 18 | 19 | 20 | 21 | 22 | 23 |
| α-haemolysis | + | + | + | V | − | + | + | + | + |
| Oxidase | + | + | + | + | + | + | V | + | + |
| Catalase | V | + | + | + | − | (−) | + | V | − |
| Alkaline phosphatase | − | − | + | − | (+) | − | − | − | − |
| γ-Glutamyltranspeptidase | − | − | NA | − | NA | NA | NA | − | − |
| Urease production | − | − | − | V | − | − | − | V* | − |
| Hippurate hydrolysis | + | + | − | − | − | − | − | − | − |
| Indoxy acetate hydrolysis | + | + | − | − | − | + | − | − | + |
| Nitrate reduction | − | + | + | + | − | + | + | + | + |
| Selenite reduction | − | + | + | + | − | + | + | + | + |
| TTC reduction | V | + | NA | + | − | − | − | − | V |
| H₂S on TSI agar | − | − | − | − | + | − | V | + | − |
| Growth 25° C. microaerobic | − | − | − | − | − | − | − | − | − |
| Growth 37° C. microaerobic | + | + | + | + | + | − | V | + | + |
| Growth 41.5° C. microaerobic | − | + | + | + | + | (−) | V | + | + |
| Growth 37° C. anaerobic | − | − | + | − | + | + | + | + | − |
| MCCDA | + | + | NA | + | + | − | + | (+) | + |
| 1% Glycine | (−) | + | − | + | V | + | V | + | + |
| 2% NaCl | − | − | − | (+) | + | V | + | + | − |
| 1% Bile | + | + | −** | + | + | − | − | V | + |
| Requirement for H2 | − | − | − | − | + | + | + | − | − |
| Nalidixic acid resistance | − | − | + | V | (+) | (+) | − | (+) | − |
| Cephalothin resistance | − | + | + | + | − | − | − | − | (−) |
| H₂S on Cysteine agar\hat{ }\hat{ }\hat{ } | NA | + | NA | + | NA | NA | NA | + | + |
| Anaerobic growth on TMAO | − | − | NA | + | − | NA | NA | +, V*** | − |
| Aerobic growth | − | − | NA | − | − | NA | NA | − | − |
| Glucose oxidation | − | − | − | − | − | − | − | − | − |
| Glucose fermentation | − | − | − | − | − | − | − | − | − |
| H₂S in FPB medium | − | V | NA | −(w)\hat{ } | − | NA | NA | +/V\hat{ }\hat{ } | − |

(notes)
+, 90-100% of strains positive
−, 0-10% of strains positive
(+), 75-89% strains positive
(−), 11-25% of strains positive
V, 26-74% of strains positive
w, weak activity
NA, no data available
MCCDA, modified charcoal cefoperazone deoxycholate agar
TTC, triphenyl tetrazolium chloride
TMAO, tri-methylamine N-oxide
*Strains of biovar parauerolyticus are urease positive other strains are urease negative, On et al. (Int. J. Syst. Evol. Microbiol, 1998, Vol 48, pp 195-206)
**(2% bile result from Vandamme et al. (2005), Bergeys Manual of Systematic Bacteriology (2005, 2^{nd} Ed, Volume 2, Part C, pp1145-1164, Springer, New York, USA),
***Most biovars are positive, sputorm biovar variable (Vandamme et al. (2005 Bergeys Manual of Systematic Bacteriology (2005, 2^{nd} Ed, Volume 2, Part C, pp1145-1164, Springer, New York, USA),
\hat{ }C. lari is weak reaction Topley and Wilsons Microbiology and Microbial Infections (9th Ed, 1998, Volume 2, Systematic Bacteriology, Chap 54, pp 1239, Arnold, London)
\hat{ }\hat{ }Most biovars are positive, fecalis biovar is variable Topley and Wilsons Microbiology and Microbial Infections (9th Ed, 1998, Volume 2, Systematic Bacteriology, Chap 54, pp 1239, Arnold, London).
\hat{ }\hat{ }\hat{ }Reference data for this test was obtained from the Animal Health and Veterinary Laboratories (AHVLA) standard operating procedure (SOP BAC89) and AHVLA studies.

TABLE 6
Distribution of MIC, MIC₅₀ and MIC₉₀ values for Campylobacter novel strains against 14 antimicrobials

| | Distribution (%) of MIC (μg/ml) | | | | | | | | | | | | MIC₅₀ | MIC₉₀ |
|---|---|---|---|---|---|---|---|---|---|---|---|---|---|---|
| Antibiotics | 0.03 | 0.06 | 0.13 | 0.25 | 0.50 | 1 | 2 | 4 | 8 | 16 | 32 | 64 | (μg/ml) | (μg/ml) |
| Ampicillin | 0 | 0 | 0 | 13.04 | 26.09 | 34.78 | 26.09 | 0 | 0 | 0 | 0 | 0 | 1 | 2 |
| Cefoperazone | 0 | 0 | 0 | 0 | 0 | 0 | 0 | 0 | 0 | 8.70 | 8.70 | 82.61 | 64 | 64 |
| Cephalothin | 0 | 0 | 0 | 0 | 0 | 0 | 0 | 0 | 8.70 | 39.13 | 39.13 | 13.04 | 32 | 32 |
| Chloramphenicol | 0 | 0 | 0 | 0 | 0 | 17.39 | 17.39 | 52.17 | 13.04 | 0 | 0 | 0 | 4 | 8 |
| Ciprofloxacin | 4.35 | 95.65 | 0 | 0 | 0 | 0 | 0 | 0 | 0 | 0 | 0 | 0 | 0.06 | 0.06 |
| Colistin | 0 | 0 | 0 | 0 | 0 | 0 | 0 | 8.70 | 73.91 | 4.35 | 13.04 | 0 | 8 | 32 |
| Ertythromycin | 0 | 0 | 0 | 0 | 26.09 | 52.17 | 21.74 | 0 | 0 | 0 | 0 | 0 | 1 | 2 |
| Gentamycin | 0 | 0 | 0 | 4.35 | 95.65 | 0 | 0 | 0 | 0 | 0 | 0 | 0 | 0.50 | 0.50 |
| Kanamycin | 0 | 0 | 0 | 0 | 0 | 0 | 8.70 | 82.61 | 8.70 | 0 | 0 | 0 | 4 | 4 |
| Nalidixic ac | 0 | 0 | 0 | 0 | 0 | 0 | 0 | 56.52 | 43.48 | 0 | 0 | 0 | 4 | 8 |
| Polymyxin B | 0 | 0 | 0 | 0 | 4.35 | 0 | 0 | 21.74 | 56.52 | 8.70 | 8.70 | 0 | 8 | 16 |
| Streptomycin | 0 | 0 | 4.35 | 0 | 21.74 | 65.22 | 8.70 | 0 | 0 | 0 | 0 | 0 | 1 | 1 |
| Tetracycline | 0 | 0 | 0 | 21.74 | 17.39 | 34.78 | 0 | 0 | 0 | 4.35 | 4.35 | 17.39 | 1 | 64 |
| Tiamulin | 0 | 0 | 0 | 4.35 | 4.35 | 13.04 | 78.26 | 0 | 0 | 0 | 0 | 0 | 2 | 2 |

### DISCUSSION

An infectious and bacterial aetiology is suggested for SLS/SLD by the epidemiology, reduction in mortality following treatment with appropriate antibiotics (Burch (2005) Veterinary Record vol. 157 p 528) and the gross and microscopic pathology.

The growth, colonial morphology and biochemistry of the isolates reported here are comparable with the vibrio-like organism isolated from AVH described by Peckham (1958, Avian Dis. vol. 2 p 348-358). However, whether these two conditions have a common aetiology is unlikely to be resolved in the absence of an isolate of the vibrio-like organism from AVH.

Based on 16S rRNA analysis the SLS/SLD isolates represent a previously undescribed lineage within the Campylobacter genus. The novel isolates are similar to a number of Campylobacter species particularly C. jejuni, C. insulaenigrae and C. subantarcticus and but do not sit comfortably within any existing species. While a 97% similarity level for bacterial speciation using 16S rRNA gene sequence has been proposed (Stackbrandt & Goebel (1994) Int. J. Syst. Bacteriol. vol. 44 p 846-849) there is no agreement on a universal cut-off for bacterial species delineation, partially reflecting the fact that different bacterial species are likely to evolve at different rates (Woo et al (2009) J. Med. Microbiol. vol. 58 p 1030-1036). Many studies have applied figures higher than 97% for bacterial species delineation. For example three recently described Campylobacter species, C. insulanigrae, C. volucris and C. subantarcticus (Debruyne et al. (2010) Int. J. Syst. Evol. Microbiol. vol. 60 p 815-819; Debruyne et al. (2010) Int. J. Syst. Evol. Microbiol. vol. 60 p 1870-1875; Foster et al. (2004) Int. J. Syst. Evol. Microbiol. vol. 54 p 2369-2373) all display higher levels of 16S rRNA sequence similarity to extant Campylobacter species than do the SLS/SLD isolates. Based on these observations and the unique phenotype of the present isolates, the isolates merit description as a new Campylobacter species.

The isolation and characterisation of this novel organism enables the detection of SLS/SLD in a sample, for example obtained as part of a routine screening procedure within an avian flock.

1. A polynucleotide comprising a sequence 1290 nucleotides in length which is at least 98.5% identical to SEQ ID NO:1, or a complementary sequence thereof.

2-3. (canceled)

4. A polynucleotide which is an RNA sequence encoded by the polynucleotide of claim 1.

5. A polynucleotide at least 6 nucleotides in length, capable of specifically hybridising to the polynucleotide of claim 1, which is not capable of hybridising to a polynucleotide not of claim 1.

6. The polynucleotide of claim 5 capable of hybridising to a region of SEQ ID NO:1 comprising one or more of the nucleotides 168T, 196A, 375T, 554A, 558T, 637A, 1152A, 1195T or 1196T, wherein the numbers correspond to the nucleotide positions in SEQ ID NO:1.

7. The polynucleotide of claim 5 capable of hybridising to a region of SEQ ID NO:1 comprising one or more of SEQ ID NOs:6-14 or 16.

8. An amplification system comprising at least one amplification primer and at least one polynucleotide of claim 5.

9. A bacterial cell comprising the polynucleotide sequence of claim 1 or deposited as ECACC Deposit Reference 12022102.

10. (canceled)

11. The cell of claim 9 which exhibits one or more of the following characteristics:
a. oxidase activity;
b. catalase activity;
c. microaerobic growth at about 37.0° C. and/or about 41.5° C.;
d. susceptibility to nalidixic acid;
e. γ-glutamyltranspeptidase activity;
f. alkaline phosphatase activity;
g. very weak or no ability to hydrolyse hippurate;
h. very weak or no ability to hydrolyse indoxyl acetate;
i. motile curved bacteria under microscopic examination; and/or
j. unable to grow in aerobic conditions.

12. The cell of claim 9 which is inactivated or is an attenuated live cell.

13. An antibody or antibody mimetic capable of binding to the polynucleotide of claim 1.

14-15. (canceled)

16. A kit comprising the polynucleotide of claim 1.

17. A method of detecting the presence of a bacterial cell comprising the polynucleotide sequence of claim 1 or deposited as ECACC Deposit Reference 12022102 in a sample, the method comprising
detecting the presence in the sample of the polynucleotide of claim 1 in the sample.

18. (canceled)

19. A method of diagnosing SLS/SLD in an animal comprising obtaining a biological sample from the animal and detecting the presence of the polynucleotide of claim 1 in the sample.

20. A cell comprising the antibody or antibody mimetic of claim 13.

21. A vaccine composition comprising the cell of claim 9, or an immunogenic portion of such a cell.

22-23. (canceled)

24. A method of vaccinating an animal against developing SLS/SLD or against infection by a bacterial cell comprising a polynucleotide comprising a sequence 1290 nucleotides in length which is at least 98.5% identical to SEQ ID NO:1, or a complementary sequence thereof, the method comprising administering an effective amount of the vaccine composition of claim 21.

25. A method of preparing the vaccine composition of claim 21 comprising attenuating or inactivating a bacterial cell comprising a polynucleotide comprising a sequence 1290 nucleotides in length which is at least 98.5% identical to SEQ ID NO:1, or a complementary sequence thereof and, optionally, mixing the resulting cell with a pharmaceutically or veterinarily acceptable carrier.

26. (canceled)

27. A method of isolating a Campylobacter strain from a test sample, comprising a step of spreading an amount of a mixed sample obtained from the test sample onto a solid medium comprising sheep's blood, wherein the sample has not previously been subjected to an earlier step of spreading onto a solid medium which comprises sheep's blood.

28-30. (canceled)

31. A method of obtaining the polynucleotide of claim 5, the method comprising designing a sequence of nucleotides which will specifically hybridise to SEQ ID NO:1 and/or 5, which will not specifically hybridise to a polynucleotide which is not SEQ ID NO:1 or 5.

32. An antibody or antibody mimetic capable of binding to the cell of claim 9.

33. A method of detecting the presence of a bacterial cell comprising a polynucleotide comprising a sequence 1290 nucleotides in length which is at least 98.5% identical to SEQ ID NO:1, or a complementary sequence thereof in a sample, the method comprising detecting the presence in the sample of the antibody of claim 13.

34. A method of detecting the presence of a bacterial cell comprising a polynucleotide comprising a sequence 1290 nucleotides in length which is at least 98.5% identical to SEQ ID NO:1, or a complementary sequence thereof in a sample, the method comprising contacting the sample with the antibody or antibody mimetic of claim 32 and detecting the antibody or antibody mimetic binding to the cell, wherein detection of binding is indicative of the presence of the cell in the sample.


SEQUENCE LISTING (18 sequences)

SEQ ID NO: 1 - DNA, 1290 bp, Campylobacter species
agcttgctagaagtggattagtggcgcacgggtgagtaaggtatagttaatctgccctacacaagaggacaacagttggaaacgactgctaatactctatactcctacttaacacaagttaagtagggaaagttttttcggtgtaggatgagactatatagtatcagttagttggtgaggtaatggctcaccaagactatgacgcttaactggtctgagaggatgatcagtcacactggaactgagacacggtccagactcctacgggaggcagcagtagggaatattgcgcaatgggggaaaccctgacgcagcaacgccgcgtggaggatgacacttttcggagcgtaaactccttttcttagggaagaattttgacggtacctaaggaataagcaccggctaactccgtgccagcagccgcggtaatacggagggtgcaagcgttactcggaatcactgggcgtaaagggcgcgtaggcggattatcaagtctcttgtgaaatctaatggcttaaccattaaactgcttgggaaactgataatctagagtaaggtagaggcagatggaattggtggtgtaggggtaaaatccgtagatatcaccaagaatacccattgcgaaggcgatctgctagaacttaactgacgctaaggcgcgaaagcgtggggagcaaacaggattagataccctggtagtccacgccctaaacgatgtacactagttgttggggtgctagtcatctcagtaatgcagctaacgcattaagtgtaccgcctggggagtacggtcgcaagattaaaactcaaaggaatagacggggacccgcacaagcggtggagcatgtggtttaattcgaagatacgcgaagaaccttacctgggcttgatatcctaagaaccttatagagatatgagggtgctagcttgctagaacttagagacaggtgctgcacggctgtcgtcagctcgtgtcgtgagatgttgggttaagtcccgcaacgagcgcaacccacgtatttagttgctaacagtttggctgagcactctaaatagactgccttcgtaaggaggaggaaggtgtggacgacgtcaagtcatcatggcccttatgcccagggcgacacacgtgctacaatggcatatacaataagacgcaataccgcaaggtggagcaaatctataaaatatgtcttagttcggattgttctctgcaactcgagagcatgaagccggaatcgctagtaatcgtagatcagccatgctacggtgaatacgttcccgggtctt

SEQ ID NO: 2 - DNA, 1436 bp, Campylobacter species
agtgaacgctggcggcgtgcctaatacatgcaagttgaacgatgaaacttctagcttgctagaagtggattagtggcgcacgggtgagtaaggtatagttaatctgccctacacaagaggacaacagttggaaacgactgctaatactctatactcctacttaacacaagttaagtagggaaagttttttcggtgtaggatgagactatatagtatcagttagttggtgaggtaatggctcaccaagactatgacgcttaactggtctgagaggatgatcagtcacactggaactgagacacggtccagactcctacgggaggcagcagtagggaatattgcgcaatgggggaaaccctgacgcagcaacgccgcgtggaggatgacacttttcggagcgtaaactccttttcttagggaagaattttgacggtacctaaggaataagcaccggctaactccgtgccagcagccgcggtaatacggagggtgcaagcgttactcggaatcactgggcgtaaagggcgcgtaggcggattatcaagtctcttgtgaaatctaatggcttaaccattaaactgcttgggaaactgataatctagagtaaggtagaggcagatggaattggtggtgtaggggtaaaatccgtagatatcaccaagaatacccattgcgaaggcgatctgctagaacttaactgacgctaaggcgcgaaagcgtggggagcaaacaggattagataccctggtagtccacgccctaaacgatgtacactagttgttggggtgctagtcatctcagtaatgcagctaacgcattaagtgtaccgcctggggagtacggtcgcaagattaaaactcaaaggaatagacggggacccgcacaagcggtggagcatgtggtttaattcgaagatacgcgaagaaccttacctgggcttgatatcctaagaaccttatagagatatgagggtgctagcttgctagaacttagagacaggtgctgcacggctgtcgtcagctcgtgtcgtgagatgttgggttaagtcccgcaacgagcgcaacccacgtatttagttgctaacagtttggctgagcactctaaatagactgccttcgtaaggaggaggaaggtgtggacgacgtcaagtcatcatggcccttatgcccagggcgacacacgtgctacaatggcatatacaataagacgcaataccgcaaggtggagcaaatctataaaatatgtcttagttcggattgttctctgcaactcgagagcatgaagccggaatcgctagtaatcgtagatcagccatgctacggtgaatacgttcccgggtcttgtactcaccgcccgtcacaccatgggagttgatttcactcgaagccggaatactaaaatagttaccgtccacagtggaatcagcgactggggtg

SEQ ID NO: 3 - DNA, 18 bp, Artificial sequence
  Amplification primer
agtttgatcctggctcag

SEQ ID NO: 4 - DNA, 20 bp, Artificial sequence
  Amplification primer
acggtaccttgttacgactt

SEQ ID NO: 5 - DNA, 1290 bp, Campylobacter species
agcttgctagaagtggattagtggcgcacgggtgagtaaggtatagttaatctgccctacacaagaggacaacagttggaaacgactgctaatactctatactcctacttaacacaagttaagtagggaaagttttttcggtgtaggatgagactatatagtatcagttagttggtgaggtaatggctcaccaagactatgacgcttaactggtctgagaggatgatcagtcacactggaactgagacacggtccagactcctacgggaggcagcagtagggaatattgcgcaatgggggaaaccctgacgcagcaacgccgcgtggaggatgacacttttcggagcgtaaactccttttcttagggaagaattttgacggtacctaaggaataagcaccggctaactccgtgccagcagccgcggtaatacggagggtgcaagcgttactcggaatcactgggcgtaaagggcgcgtaggcggattatcaagtctcttgtgaaatctaatggcttaaccattaaactgcttgggaaactgataatctagagtaaggtagaggcagatggaattggtggtgtaggggtaaaatccgtagatatcaccaagaatacccattgcgaaggcgatctgctagaacttaactgacgctaaggcgcgaaagcgtggggagcaaacaggattagataccctggtagtccacgccctaaacgatgtacactagttgttggggtgctagtcatctcagtaatgcagctaacgcattaagtgtaccgcctggggagtacggtcgcaagattaaaactcaaaggaatagacggggacccgcacaagcggtggagcatgtggtttaattcgaagatacgcgaagaaccttacctgggcttgatatcctaagaaccttatagagatatgagggtgctagcttgctagaacttagagacaggtgctgcacggctgtcgtcagctcgtgtcgtgagatgttgggttaagtcccgcaacgagcgcaacccacgtatttagttgctaacagtttggctgagcactctaaatagactgccttcgtaaggaggaggaaggtgtggacgacgtcaagtcatcatggcccttatgcccagggcgacacacgtgctacaatgacatatacaataagacgcaataccgcaaggtggagcaaatctataaaatatgtcttagttcggattgttctctgcaactcgagagcatgaagccggaatcgctagtaatcgtagatcagccatgctacggtgaatacgttcccgggtctt

SEQ ID NO: 6 - DNA, 10 bp, Artificial Sequence
  Probe hybridisation sequence
tcagttagtt

SEQ ID NO: 7 - DNA, 10 bp, Artificial Sequence
  Probe recognition sequence
caagactatg

SEQ ID NO: 8 - DNA, 10 bp, Artificial Sequence
  Probe hybridisation sequence
aattttgacg

SEQ ID NO: 9 - DNA, 10 bp, Artificial Sequence
  Probe hybridisation sequence
gagtaaggta

SEQ ID NO: 10 - DNA, 10 bp, Artificial Sequence
  Probe hybridisation sequence
aaggtagagg

SEQ ID NO: 11 - DNA, 10 bp, Artificial Sequence
  Probe recognition sequence
agtaaggtag

SEQ ID NO: 12 - DNA, 10 bp, Artificial Sequence
  Probe hybridisation sequence
tgctagaact

SEQ ID NO: 13 - DNA, 10 bp, Artificial Sequence
  Probe hybridisation sequence
caataagacg

SEQ ID NO: 14 - DNA, 12 bp, Artificial Sequence
  Probe hybridisation sequence
gcatatacaata

SEQ ID NO: 15 - DNA, 12 bp, Artificial Sequence
  Probe hybridisation sequence
acatatacaata

SEQ ID NO: 16 - DNA, 10 bp, Artificial Sequence
  Probe hybridisation sequence
tgtcttagtt

SEQ ID NO: 17 - DNA, 52 bp, Artificial Sequence
  Primer hybridisation region
agtgaacgctggcggcgtgcctaatacatgcaagttgaacgatgaaacttct

SEQ ID NO: 18 - DNA, 94 bp, Artificial Sequence
  Primer hybridisation region
gtactcaccgcccgtcacaccatgggagttgatttcactcgaagccggaatactaaaatagttaccgtccacagtggaatcagcgactggggtg